(19) United States
(12) Patent Application Publication
(10) Pub. No.: US 2006/0030557 A1
(43) Pub. Date: Feb. 9, 2006

(54) Substituted pyridines having antiangiogenic activity

(76) Inventors: Fortuna Haviv, Deerfield, IL (US); Michael F. Bradley, Covington, WA (US); Jack Henkin, Highland Park, IL (US); Jurgen Dinges, Grayslake, IL (US); Daryl R. Sauer, Trevor, WI (US); Lawrence Kolaczkowski, Gurnee, IL (US); Anil Vasudevan, Gurnee, IL (US); David C. Park, Lake Bluff, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/244,481

(22) Filed: Oct. 6, 2005

Related U.S. Application Data
(62) Division of application No. 10/387,367, filed on Mar. 12, 2003.
Application No. 10/387,367 is a continuation-in-part of application No. 10/244,987, filed on Sep. 17, 2002, now abandoned.
Application No. 10/244,987 is a continuation-in-part of application No. 10/116,971, filed on Apr. 5, 2002, now abandoned.

(51) Int. Cl. A61K 31/551; A61K 31/541; C07D 417/02; C07D 403/02

(52) U.S. Cl. 514/218; 514/227.5; 540/575; 544/60

(57) ABSTRACT

Compounds having the formula 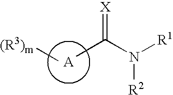 are angiogenesis inhibitors. Also disclosed are compositions containing the compounds, methods of making the compounds, and methods of treatment using the compounds.

### CROSS-REFERENCE TO RELATED APPLICATIONS

This application is a continuation-in-part of U.S. patent application Ser. No. 10/244,987, filed on Sep. 17, 2002, which is a continuation-in-part of U.S. patent application Ser. No. 10/116,971, filed on Apr. 5, 2002. Both of these applications are hereby incorporated by reference.

### TECHNICAL FIELD

The present invention relates to novel compounds having activity useful for treating conditions which arise from or are exacerbated by angiogenesis, pharmaceutical compositions comprising the compounds, methods of treatment using the compounds, methods of inhibiting angiogenesis, and methods of treating cancer.

### BACKGROUND OF THE INVENTION

Angiogenesis is the fundamental process by which new blood vessels are formed and is essential to a variety of normal body activities (such as reproduction, development and wound repair). Although the process is not completely understood, it is believed to involve a complex interplay of molecules which both stimulate and inhibit the growth of endothelial cells, the primary cells of the capillary blood vessels. Under normal conditions these molecules appear to maintain the microvasculature in a quiescent state (i.e., one of no capillary growth) for prolonged periods that may last for weeks, or in some cases, decades. However, when necessary, such as during wound repair, these same cells can undergo rapid proliferation and turnover within as little as five days.

Although angiogenesis is a highly regulated process under normal conditions, many diseases (characterized as “angiogenic diseases”) are driven by persistent unregulated angiogenesis. Otherwise stated, unregulated angiogenesis may either cause a particular disease directly or exacerbate an existing pathological condition. For example, the growth and metastasis of solid tumors have been shown to be angiogenesis-dependent. Based on these findings, there is a continuing need for compounds which demonstrate antiangiogenic activity due to their potential use in the treatment of various diseases such as cancer.

### SUMMARY OF THE INVENTION

In its principle embodiment the present invention provides a compound of formula (I) 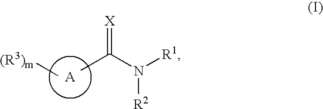
or a therapeutically acceptable salt thereof, wherein
A is an aromatic six-membered ring containing from one to three nitrogen atoms wherein the remaining atoms are carbon;
R¹ and R², together with the nitrogen atom to which they are attached, form a five- to eight-membered ring containing an additional zero to two heteroatoms selected from the group consisting of nitrogen, oxygen, and sulfur, wherein the ring can be optionally substituted with one, two, or three substituents independently selected from the group consisting of alkoxyalkyl, alkoxycarbonyl, alkyl, alkylcarbonyl, amino, aminocarbonyl, aryl, arylalkoxycarbonyl, arylalkyl, carboxy, formyl, haloalkyl, heterocycle, (heterocycle)alkyl, hydroxy, hydroxyalkoxyalkyl, hydroxyalkyl, and spiroheterocycle;
R³ at each occurance is independently selected from the group consisting of alkenyl, alkoxy, alkoxyalkyl, alkoxycarbonyl, alkyl, alkylcarbonyl, alkylsulfanyl, amino, aminocarbonyl, aryl, arylalkyl, aryloxy, cyano, cyanoalkyl, cycloalkyl, (cycloalkyl)alkyl, halo, haloalkyl, heterocycle, hydroxy, hydroxyalkyl, and nitro;
X is selected from the group consisting of O, S, and CH₂; and
m is 0-4.

In a preferred embodiment the present invention provides the compound of formula (I) wherein X is O and A is a six-membered aromatic ring containing two nitrogen atoms wherein the remaining atoms are carbon.

In another preferred embodiment the present invention provides the compound of formula (I) wherein X is O and A is a six-membered aromatic ring containing one nitrogen atom wherein the remaining atoms are carbon.

In another preferred embodiment the present invention provides a compound of formula (II) 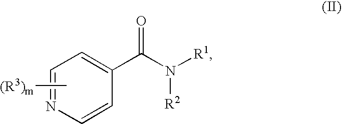
or a therapeutically acceptable salt thereof, wherein
R¹, R², R³, and m are as previously described.

In another preferred embodiment the present invention provides a compound of formula (III) 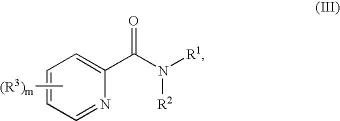
or a therapeutically acceptable salt thereof, wherein
R¹, R², R³, and m are as described above.

In another preferred embodiment the present invention provides a compound of formula (IV) 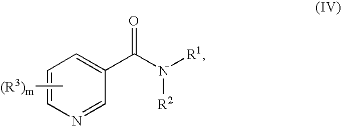
or a therapeutically acceptable salt thereof, wherein
R¹, R², R³, and m are as described above.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a ring selected from the group consisting of diazepanyl, thiomorpholinyl, morpholinyl, piperazinyl, piperidinyl, and pyrrolidinyl.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a diazepanyl ring.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a thiomorpholinyl ring.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a piperazinyl ring.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a piperidinyl ring.

In a more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a piperidinyl ring, wherein the piperidinyl ring is unsubstituted or is substituted with one substituent selected from the group consisting of hydroxy and spiroheterocycle.

In another more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a piperidinyl ring, wherein the piperidinyl ring is substituted with one substituent selected from the group consisting of alkoxycarbonyl, aminocarbonyl, arylalkyl, and heterocycle.

In another more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a piperidinyl ring, wherein the piperidinyl ring is substituted with an alkyl group.

In another preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a pyrrolidinyl ring.

In a more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a pyrrolidinyl ring, wherein the pyrrolidinyl ring is unsubstituted or substituted with one substituent selected from the group consisting of alkoxyalkyl, alkoxycarbonyl, aminocarbonyl, arylalkoxycarbonyl, carboxy, heterocycle, (heterocycle)alkyl, and hydroxyalkyl.

In another more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a pyrrolidinyl ring, wherein the pyrrolidinyl ring is substituted with one substituent selected from the group consisting of amino, aryl, and arylalkyl.

In another more preferred embodiment the present invention provides a compound of formula (I) wherein X is O, A is a six-membered aromatic ring containing one nitrogen atom wherein the remaining atoms are carbon, and R¹ and R², together with the nitrogen atom to which they are attached, form a pyrrolidinyl ring, wherein the pyrrolidinyl ring is substituted with one or two alkyl groups.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, and m is 0 or 2.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, and m is 1.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, m is 1, and R³ is selected from the group consisting of alky, halo, and hydroxy.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, m is 1, and R³ is selected from the group consisting of alkyl and aryl.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, m is 1, and R³ is selected from the group consisting of cycloalkyl, (cycloalkyl)alkyl, cyanoalkyl, and heterocycle.

In another more preferred embodiment the present invention provides a compound of formula (I) wherein X is O, A is a six-membered aromatic ring containing one nitrogen atom wherein the remaining atoms are carbon, R¹ and R², together with the nitrogen atom to which they are attached, form a pyrrolidinyl ring, wherein the pyrrolidinyl ring is substituted with one or two alkyl groups, m is 1, and R³ is selected from the group consisting of hydrogen and amino.

In a particularly preferred embodiment the present invention provides a compound which is
2-methyl-5-[(2-methylpyrrolidin-1-yl)carbonyl]pyridine.

In another particularly preferred embodiment the present invention provides a compound which is
1-[(6-methylpyridin-3-yl)carbonyl]piperidine-3-carboxamide.

In another particularly preferred embodiment the present invention provides a compound which is
(3S)-N,N-dimethyl-1-[(6-methyl-3-pyridinyl)carbonyl]-3-pyrrolidinamine.

In another particularly preferred embodiment the present invention provides a compound which is
(3R)-N,N-dimethyl-1-[(6-methyl-3-pyridinyl)carbonyl]-3-pyrrolidinamine.

In another particularly preferred embodiment the present invention provides a compound which is
(3R)-1-[(6-methyl-3-pyridinyl)carbonyl]-3-piperidinecarboxamide.

In another particularly preferred embodiment the present invention provides a compound which is
(3S)-1-[(6-methyl-3-pyridinyl)carbonyl]-3-piperidinecarboxamide.

In another particularly preferred embodiment the present invention provides a compound which is
1-(4-fluorophenyl)-4-[(6-methylpyridin-3-yl)carbonyl]piperazine.

In another particularly preferred embodiment the present invention provides a compound which is
(2S)-1-[(6-methyl-3-pyridinyl)carbonyl]-2-piperidinecarboxamide.

In another particularly preferred embodiment the present invention provides a compound which is
(2R)-1-[(6-methyl-3-pyridinyl)carbonyl]-2-piperidinecarboxamide.

In another particularly preferred embodiment the present invention provides a compound which is
(3S)-1-[(5-methyl-3-pyridinyl)carbonyl]-3-piperidinecarboxamide.

In another particularly preferred embodiment the present invention provides a compound which is
(3R)-1-[(5-methyl-3-pyridinyl)carbonyl]-3-piperidinecarboxamide.

In another particularly preferred embodiment the present invention provides a compound which is
(3R)-N,N-dimethyl-1-[(5-methyl-3-pyridinyl)carbonyl]-3-pyrrolidinamine.

In another particularly preferred embodiment the present invention provides a compound which is
(3S)-N,N-dimethyl-1-[(5-methyl-3-pyridinyl)carbonyl]-3-pyrrolidinamine.

In another embodiment the present invention provides a pharmaceutical composition comprising a compound of formula (I) or a therapeutically acceptable salt thereof, in combination with a therapeutically acceptable carrier.

In another embodiment the present invention provides a method for inhibiting angiogenesis in a patient in recognized need of such treatment comprising administering to the patient a therapeutically acceptable amount of a compound of formula (I), or a therapeutically acceptable salt thereof.

In another embodiment the present invention provides a method for treating cancer in a patient in recognized need of such treatment comprising administering to the patient a therapeutically acceptable amount of a compound of formula (I), or a therapeutically acceptable salt thereof.

### DETAILED DESCRIPTION OF THE INVENTION

Compounds of the present invention comprise substituted heterocyclic compounds which are useful for the treatment of diseases which are caused or exacerbated by angiogenesis. The compounds of the invention are also useful for the treatment of cancer.

It is intended that the definition of any substituent or variable (e.g., R³) at a particular location in a molecule be independent of its definitions elsewhere in that molecule. Thus, (R³)₂ represents two R³ groups which may be the same or different.

As used herein, the singular forms “a”, “an”, and “the” include plural reference unless the context clearly dictates otherwise.

As used in the present specification the following terms have the meanings indicated:

The term “alkenyl,” as used herein, represents a straight or branched chain group of one to twelve carbon atoms derived from a straight or branched chain hydrocarbon containing at least one carbon-carbon double bond.

The term “alkoxy,” as used herein, represents an alkyl group attached to the parent molecular moiety through an oxygen atom.

The term “alkoxyalkyl,” as used herein, represents an alkyl group subsituted with at least one alkoxy group.

The term “alkoxycarbonyl,” as used herein, represents an alkoxy group attached to the parent molecular moiety through a carbonyl group.

The term “alkyl,” as used herein, represents a group of one to twelve carbon atoms derived from a straight or branched chain saturated hydrocarbon. Examples of alkyl groups include, but are not limited to, methyl, ethyl, propyl, butyl, isobutyl, 1-methylpentyl, and hexyl.

The term “alkylcarbonyl,” as used herein, represents an alkyl group attached to the parent molecular moiety through a carbonyl group.

The term “alkylsulfanyl,” as used herein, represents an alkyl group attached to the parent molecular moiety through a sulfur atom.

The term “alkylsulfonyl,” as used herein, represents an alkyl group attached to the parent molecular moiety through a sulfonyl group.

The term “amino,” as used herein, represents —NR⁹R¹⁰, wherein R⁹ and R¹⁰ are independently selected from the group consisting of hydrogen, alkenyl, alkoxyalkyl, alkoxycarbonyl, alkyl, alkylcarbonyl, aryl, arylalkyl, arylcarbonyl, arylsulfonyl, cycloalkyl, (cycloalkyl)alkyl, cycloalkylcarbonyl, heterocycle, (heterocycle)alkyl, hydroxyalkyl, and (NR^{a}R^{b})alkyl, wherein R^{a} and R^{b} are independently selected from the group consisting of hydrogen and alkyl, and wherein the aryl; the aryl part of the arylalkyl, the arylalkylcarbonyl, the arylcarbonyl, and the arylsulfonyl; the cycloalkyl; the cycloalkyl part of the (cycloalkyl)alkyl and the cycloalkylcarbonyl; the heterocycle; and the heterocycle part of the (heterocycle)alkyl can be optionally substituted with one, two, three, four, or five substituents independently selected from the group consisting of alkoxy, alkyl, alkylcarbonyl, cyano, halo, haloalkoxy, haloalkyl, hydroxy, and nitro.

The term “aminoalkyl,” as used herein, represents an alkyl group substituted with at least one amino group.

The term “aminocarbonyl,” as used herein, represents an amino group attached to the parent molecular moiety through a carbonyl group.

The term “aminosulfonyl,” as used herein, represents an amino group attached to the parent molecular moiety through a sulfonyl group.

The term “aryl,” as used herein, represents a phenyl group or a bicyclic or tricyclic fused ring system wherein one or more of the fused rings is a phenyl group. Bicyclic fused ring systems are exemplified by a phenyl group fused to a monocyclic cycloalkyl group as defined herein, a monocyclic cycloalkenyl group as defined herein, or another phenyl group. Tricyclic fused ring systems are exemplified by a bicyclic fused ring system fused to a monocyclic cycloalkyl group as defined herein, a monocyclic cycloalkenyl group as defined herein, or another phenyl group. Representative examples of aryl include, but are not limited to, anthracenyl, azulenyl, fluorenyl, indanyl, indenyl, naphthyl, phenyl, and tetrahydronaphthyl. Aryl groups having an unsaturated or partially saturated ring fused to an aromatic ring can be attached through the saturated or the unsaturated part of the group. The aryl groups of this invention can be optionally substituted with one, two, three, four, or five substituents independently selected from the group consisting of alkenyl, alkoxy, alkoxyalkyl, alkoxycarbonyl, alkyl, alkylcarbonyl, alkylsulfonyl, amino, aminoalkyl, aminocarbonyl, aminosulfonyl, a second aryl group, arylalkyl, carboxy, cyano, cyanoalkyl, cycloalkyl, (cycloalkyl)alkyl, formyl, halo, haloalkoxy, haloalkyl, heterocycle, (heterocycle)alkyl, hydroxy, hydroxyalkyl, nitro, and oxo; wherein the second aryl group; the aryl part of the arylalkyl; the heterocycle; and the heterocycle part of the (heterocycle)alkyl can be further optionally substituted with one, two, or three substituents independently selected from the group consisting of alkoxy, alkoxycarbonyl, alkyl, alkylcarbonyl, carboxy, cyano, formyl, halo, haloalkoxy, haloalkyl, hydroxy, hydroxyalkyl, nitro, and oxo.

The term “arylalkoxy,” as used herein, represents an arylalkyl group attached to the parent molecular moiety through an oxygen atom.

The term “arylalkoxycarbonyl,” as used herein, represents an arylalkoxy group attached to the parent molecular moiety through a carbonyl group.

The term “arylalkyl,” as used herein, represents an alkyl group substituted with at least one aryl group.

The term “arylcarbonyl,” as used herein, represents an aryl group attached to the parent molecular moiety through a carbonyl group.

The term “aryloxy,” as used herein, represents an aryl group attached to the parent molecular moiety through an oxygen atom.

The term “arylsulfonyl,” as used herein, represents an aryl group attached to the parent molecular moiety through a sulfonyl group.

The term “carbonyl,” as used herein, represents —C(O)—.

The term “carboxy,” as used herein, represents —CO₂H.

The term “cyano,” as used herein, represents —CN.

The term “cyanoalkyl,” as used herein, represents an alkyl group substituted with at least one cyano group.

The term “cycloalkenyl,” as used herein, represents a non-aromatic ring system having three to ten carbon atoms and one to three rings, wherein at least one ring is a five-membered ring with one double bond, a six-membered ring with one or two double bonds, a seven- or eight-membered ring with one to three double bonds, or a nine- to ten-membered ring with one to four double bonds. Examples of cycloalkenyl groups include, but are not limited to, cyclohexenyl, octahydronaphthalenyl, and norbornylenyl.

The term “cycloalkyl,” as used herein, represents a saturated ring system having three to twelve carbon atoms and one to three rings. Examples of cycloalkyl groups include, but are not limited to, cyclopropyl, cyclopentyl, bicyclo(3.1.1)heptyl, adamantyl, and bicyclo[2.2.1]heptyl. The cycloalkyl groups of this invention can be optionally substituted with one, two, three, four, or five substituents independently selected from the group consisting of alkoxy, alkoxycarbonyl, alkyl, amino, aminoalkyl, halo, haloalkoxy, haloalkyl, hydroxy, and nitro.

The term “(cycloalkyl)alkyl,” as used herein, represents an alkyl group substituted with at least one cycloalkyl group.

The term “cycloalkylcarbonyl,” as used herein, represents a cycloalkyl group attached to the parent molecular moiety through a carbonyl group.

The term “formyl,” as used herein, represents —CHO.

The terms “halo,” and “halogen,” as used herein, represent F, Cl, Br, and I.

The term “haloalkoxy,” as used herein, represents an alkoxy group substituted with one, two, three, or four halogen atoms.

The term “haloalkyl,” as used herein, represents an alkyl group substituted by one, two, three, or four halogen atoms.

The term “heteroalkenylene,” as used herein, represents an unsaturated group of two to six atoms containing one or two heteroatoms independently selected from the group consisting of nitrogen, oxygen, and sulfur, wherein the remaining atoms are carbon. The heteroalkylene groups of the present invention can be attached to the parent molecular moiety through the carbon atoms or the heteroatoms in the chain.

The term “heteroalkylene,” as used herein, represents a saturated group of two to six atoms containing one or two heteroatoms independently selected from the group consisting of nitrogen, oxygen, and sulfur, wherein the remaining atoms are carbon. The heteroalkylene groups of the present invention can be attached to the parent molecular moiety through the carbon atoms or the heteroatoms in the chain.

The term “heterocycle,” as used herein, represents a monocyclic, bicyclic, or tricyclic ring system wherein one or more rings is a four-, five-, six-, or seven-membered ring containing one, two, or three heteroatoms independently selected from the group consisting of nitrogen, oxygen, and sulfur. Monocyclic ring systems are exemplified by any 3- or 4-membered ring containing a heteroatom independently selected from the group consisting of oxygen, nitrogen and sulfur; or a 5-, 6- or 7-membered ring containing one, two or three heteroatoms wherein the heteroatoms are independently selected from the group consisting of nitrogen, oxygen and sulfur. The 3- and 4-membered rings have no double bonds, the 5-membered ring has from 0-2 double bonds and the 6- and 7-membered rings have from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, and trithiane. Bicyclic ring systems are exemplified by any of the above monocyclic ring systems fused to phenyl ring, a monocyclic cycloalkyl group as defined herein, a monocyclic cycloalkenyl group, as defined herein, or another monocyclic heterocycle ring system. Representative examples of bicyclic ring systems include but are not limited to, benzimidazole, benzothiazole, benzothiophene, benzoxazole, benzofuran, benzopyran, benzothiopyran, benzodioxine, 1,3-benzodioxole, cinnoline, dihydrobenzimidazole, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, and thiopyranopyridine. Tricyclic rings systems are exemplified by any of the above bicyclic ring systems fused to a phenyl ring, a monocyclic cycloalkyl group as defined herein, a monocyclic cycloalkenyl group as defined herein, or another monocyclic heterocycle ring system. Representative examples of tricyclic ring systems include, but are not limited to, acridine, carbazole, carboline, dibenzofuran, dibenzothiophene, naphthofuran, naphthothiophene, oxanthrene, phenazine, phenoxathiin, phenoxazine, phenothiazine, thianthrene, thioxanthene, and xanthene. Heterocycle groups can be attached to the parent molecular moiety through a carbon atom or a nitrogen atom in the group.

The heterocycle groups of the present invention can be optionally substituted with one, two, three, four, or five substituents independently selected from the group consisting of alkenyl, alkoxy, alkoxyalkyl, alkoxycarbonyl, alkyl, alkylcarbonyl, alkylsulfonyl, amino, aminoalkyl, aminocarbonyl, aminosulfonyl, aryl, arylalkyl, carboxy, cyano, cyanoalkyl, cycloalkyl, (cycloalkyl)alkyl, formyl, halo, haloalkoxy, haloalkyl, a second heterocycle, (heterocycle)alkyl, hydroxy, hydroxyalkyl, nitro, and oxo; wherein the aryl, the aryl part of the arylalkyl, the second heterocycle; and the heterocycle part of the (heterocycle)alkyl can be further optionally substituted with one, two, three, four, or five substituents independently selected from the group consisting of alkoxy, alkoxycarbonyl, alkyl, alkylcarbonyl, carboxy, cyano, formyl, halo, haloalkoxy, haloalkyl, hydroxy, hydroxyalkyl, nitro, and oxo.

The term “(heterocycle)alkyl,” as used herein, represents an alkyl group substituted with at least one heterocycle group.

The term “heterocyclecarbonyl,” as used herein, represents a heterocycle group attached to the parent molecular moiety through a carbonyl group.

The term “hydroxy,” as used herein, represents —OH.

The term “hydroxyalkyl,” as used herein, represents an alkyl group substituted with at least one hydroxy group.

The term “nitro,” as used herein, represents —NO₂.

The term “—NR^{a}R^{b},” as used herein, represents two groups, R^{a} and R^{b}, which are attached to the parent molecular moiety through a nitrogen atom. R^{a} and R^{b} are independently selected from the group consisting of hydrogen and alkyl.

The term “(NR^{a}R^{b})alkyl,” as used herein, represents an alkyl group substituted with at least one —NR^{a}R^{b} group.

The term “oxo,” as used herein, represents ═O.

The term “spiroheterocycle,” as used herein, represents a heteroalkenylene or heteroalkylene group in which both ends of the heteroalkenylene or heteroalkylene group are attached to the same carbon of the parent molecular moiety to form a bicyclic group. The spiroheterocycle groups of the present invention can be optionally substituted with one or two alkyl groups.

The term “sulfonyl,” as used herein, represents —SO₂—.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, or mixtures thereof, which possess the ability to inhibit angiogenesis and/or treat cancer. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

In accordance with methods of treatment and pharmaceutical compositions of the invention, the compounds can be administered alone or in combination with other chemotherapeutic agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Preferred compounds of the present invention are compounds of formula (I) where A is an aromatic six-membered ring containing one nitrogen atom wherein the remaining atoms are carbon.

Determination of Biological Activity

In Vitro Assay for Angiogenic Activity

The human microvascular endothelial (HMVEC) migration assay was run according to the procedure of S. S. Tolsma, O. V. Volpert, D. J. Good, W. F. Frazier, P. J. Polyerini and N. Bouck, J. Cell Biol. 122, 497-511 (1993).

The HMVEC migration assay was carried out using Human Microvascular Endothelial Cells-Dermal (single donor) and Human Microvascular Endothelial Cells, (neonatal). The BCE or HMVEC cells were starved overnight in DME containing 0.01% bovine serum albumin (BSA). Cells were then harvested with trypsin and resuspended in DME with 0.01% BSA at a concentration of 1.5×10⁶ cells per mL. Cells were added to the bottom of a 48 well modified Boyden chamber (Nucleopore Corporation, Cabin John, Md.). The chamber was assembled and inverted, and cells were allowed to attach for 2 hours at 37° C. to polycarbonate chemotaxis membranes (5 μm pore size) that had been soaked in 0.01% gelatin overnight and dried. The chamber was then reinverted, and test substances (total volume of 50 μL), including activators, 15 ng/mL bFGF/VEGF, were added to the wells of the upper chamber. The apparatus was incubated for 4 hours at 37° C. Membranes were recovered, fixed and stained (Diff Quick, Fisher Scientific) and the number of cells that had migrated to the upper chamber per 3 high power fields counted. Background migration to DME+0.1 BSA was subtracted and the data reported as the number of cells migrated per 10 high power fields (400×) or, when results from multiple experiments were combined, as the percent inhibition of migration compared to a positive control.

Representative compounds described in Examples 1 to 183 inhibited human endothelial cell migration in the above assay by at least 45% when tested at a concentration of 1 nM. Preferred compounds inhibited human endothelial cell migration by about 70 to about 95% when tested at a concentration of 1 nM.

Many diseases (characterized as “angiogenic diseases”) are driven by persistent unregulated angiogenesis. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. Growth and metastasis of solid tumors are also angiogenesis-dependent (Folkman, J., Cancer Res., 46: 467-473 (1986), Folkman, J., J. Natl. Cancer Inst., 82: 4-6 (1989)). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites, such as the liver, the lung, and the bones (Weidner, N., et. al., N. Engl. J. Med., 324(1): 1-8 (1991)).

The compounds of the invention, including but not limited to those specified in the examples, possess antiangiogenic activity. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungicides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents. The compounds of the invention can also be useful in the treatment of the aforementioned conditions by mechanisms other than the inhibition of angiogenesis.

Further uses include the treatment and prophylaxis of autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemagiomas, and capillary proliferation within atherosclerotic plaques; Osler-Webber Syndrome; myocardial angiogenesis; plaque neovascularization; telangiectasia; hemophiliac joints; angiofibroma; and wound granulation. Other uses include the treatment of diseases characterized by excessive or abnormal stimulation of endothelial cells, including not limited to intestinal adhesions, Crohn's disease, atherosclerosis, scleroderma, and hypertrophic scars, i.e., keloids. Another use is as a birth control agent, by inhibiting ovulation and establishment of the placenta. The compounds of the invention are also useful in the treatment of diseases that have angiogenesis as a pathologic consequence such as cat scratch disease (Rochele minutesalia quintosa) and ulcers (Helicobacter pylori). The compounds of the invention are also useful to reduce bleeding by administration prior to surgery, especially for the treatment of resectable tumors.

Synthetic Methods

Abbreviations which have been used in the descriptions of the scheme and the examples that follow are: DCC for 1,3-dicyclohexylcarbodiimide; HOBT for 1-hydroxybenzotriazole; PPh₃ for triphenylphosphine, THF for tetrahydrofuran, TFA for trifluoroacetic acid, DMSO for dimethylsulfoxide, DMF for N,N-dimethylformamide, and Fmoc for N-(9-fluorenylmethoxycarbonyl).

The compounds and processes of the present invention will be better understood in connection with the following synthetic scheme which illustrates the method by which the compounds of the invention may be prepared. Starting materials can be obtained from commercial sources or prepared by well-established literature methods known to those of ordinary skill in the art. The groups A, R¹, R², and R³ are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

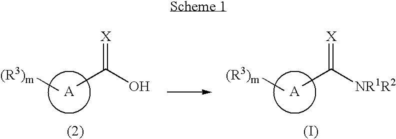

Scheme 1 shows the synthesis of compounds of formula (I). Compounds of formula (2) can be converted to the corresponding acid chloride by treatment with thionyl chloride. Examples of solvents used in this reaction include dichloromethane, chloroform, and carbon tetrachloride. The reaction is typically conducted at about −5° C. to about 30° C. for about 30 minutes to about 2 hours. The acid chloride can then be reacted with an appropriately substituted amine (HNR¹R²) in the presence of a base such as triethylamine or diisopropylethylamine to provide compounds of formula (I). Examples of solvents used in this reaction include dichloromethane, chloroform, and carbon tetrachloride. The reaction is typically run at about 0° C. to about 40° C. for about 2 to about 6 hours.

Compounds of formula (2) can also be converted to compounds of formula (I) by treatment with an appropriately subsituted amine (HNR¹R²) under coupling conditions (e.g., DCC with or without HOBT, and other reagents known to those of ordinary skill in the art).

Alternatively, compounds of formula (2) can be treated with N-hydroxysuccinimide under coupling conditions (e.g., DCC, HOBT, and other reagents known to those of ordinary skill in the art) to provide the N-hydroxysuccinimide ester which can then be reacted with the corresponding amine (HNR¹R²) to provide compounds of formula (I).

Compounds of formula (I) where R³ is halo can be coupled with an organoborane (in the presence of a base such as sodium carbonate or cesium fluoride) or an organostannane in the presence of a palladium catalyst such as Pd(PPh₃)₄ or PdCl₂(PPh₃)₂ to provide compounds where R³ is alkyl, cyanoalkyl, cycloalkyl, (cycloalkyl)alkyl, aryl, or heterocycle. Examples of solvents used in these reactions include dichloromethane, toluene, and THF. The reaction is typically conducted at about 25° C. to about 100° C. (depending on the conditions used) for about 8 to about 24 hours.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names which appeared to be consistent with ACD nomenclature.

### EXAMPLE 1

### 2-methyl-5-[(2-methylpyrrolidin-1-yl)carbonyl]pyridine

A suspension of 6-methylnicotinic acid (8.25 g, 60 mmol) in dry dichloromethane at 0° C. (90 mL) was treated with thionyl chloride (9 mL, 124 mmol), stirred for 1 hour, and concentrated under vacuum. The residue was added dropwise to a solution of 2-methylpyrrolidine (6.21 mL, 60 mmol) and triethylamine (45 mL) in dichloromethane (200 mL) at 0° C., stirred for 4 hours, and concentrated under vacuum. The concentrate was dissolved in dichloromethane, washed sequentially with saturated sodium bicarbonate, water, and brine, then dried (MgSO₄), filtered, and concentrated. The crude product was purified by flash column chromatography with dichloromethane and (99:1) dichloromethane/methanol, dissolved in diethyl ether, treated with 2 M HCl in diethyl ether (80 mL), and filtered. The filter cake was washed with diethyl ether and dried under vacuum. The solid was recrystallized from methanol/ethyl acetate/hexanes to provide the desired product (8.04 g) as the hydrochloride salt. MS m/e 205.1 (M+H)⁺; ¹H NMR (DMSO-d₆) δ 0.87 (d, 0.75H), 1.27 (d, 2.25H), 1.53-1.63 (m, 1H), 1.69-1.79 (m, 1H), 1.85-1.95 (m, 1H), 2.05-2.13 (m, 1H), 2.80 (s, 3H), 3.32-3.41 (m, 0.8H), 3.48-3.59 (m, 1.2H), 3.94-4.02 (m, 0.25H), 4.12-4.20 (m, 0.75H), 7.94 (dd, 1H), 8.52 (dd, 1H), 8.87 (d, 0.75H), 8.93 (br s, 0.25H).

### EXAMPLE 2

### 2-methyl-5-(piperidin-1-ylcarbonyl)pyridine

The desired product was prepared by substituting piperidine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 205.1 (M+H)⁺; ¹H NMR (DMSO-d₆) δ 1.39-1.65 (m, 6H), 2.55 (s, 3H), 3.27 (br s, 2H), 3.59 (br s, 2H), 7.47 (dd, 1H), 7.87 (dd, 1H), 8.56 (d, 1H).

### EXAMPLE 3

### 5-[(2-ethylpiperidin-1-yl)carbonyl]-2-methylpyridine

The desired product was prepared by substituting 2-ethylpiperidine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 233 (M+H)⁺; ¹H NMR (DMSO-d₆) δ 0.77 (br d, 3H), 1.32-1.73 (br m, 7H), 1.74-1.84 (m, 1H), 2.58 (s, 3H), 2.78 (br s, 0.5H), 3.10 (br s, 0.5H), 3.31 (br s, 0.5H), 3.51 (br s, 0.51H), 4.34 (br s, 0.51H), 4.60 (br s, 0.5H), 7.54 (dd, 1H), 7.93 (dd, 1H), 8.59 (d, 1H).

### EXAMPLE 4

### 2-methyl-5-[(4-propylpiperidin-1-yl)carbonyl]pyridine

The desired product was prepared by substituting 4-propylpiperidine for 2-methylpyrrolidine. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt MS m/e 247 (M+H)⁺; ¹H NMR (DMSO-d₆) δ 0.87 (t, 31), 1.03-1.14 (br m, 2H), 1.17-1.25 (m, 2H), 1.26-1.35 (m, 2H), 1.48-1.64 (br m, 2H), 1.69-1.80 (br s, 1H), 2.58 (s, 3H), 2.71-2.84 (br m, 1H), 2.99-3.11 (br m, 1H).

### EXAMPLE 5

### 4-[(6-methylpyridin-3-yl)carbonyl]thiomorpholine

The desired product was prepared by substituting thiomorpholine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 223 (M+H)⁺; ¹H NMR (DMSO-d₆) δ 2.56-2.74 (br m, 4H), 2.75 (s, 3H), 3.55 (br s, 2H), 3.88 (br s, 2H), 7.87 (dd, 1H), 8.36 (dd, 1H), 8.83 (d, 1H).

### EXAMPLE 6

### 8-[(6-methylpyridin-3-yl)carbonyl]-1,4-dioxa-8-azaspiro[4.5]decane

The desired product was prepared by substituting 4-piperidone ethylene ketal for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 263.1 (M+H)⁺; ¹H NMR (DMSO-d₆) δ 1.67 (br s, 4H), 2.58 (s, 3H), 3.37 (br s, 2H), 3.68 (br s, 2H), 3.91 (s, 4H); 7.54 (dd, 1H), 7.96-8.03 (m, 1H), 8.64 (d, 0.66H), 8.69 (d, 0.33H).

### EXAMPLE 7

### 1-[(5-bromopyridin-3-yl)carbonyl]-1,4-diazepane

The desired product was prepared by substituting 5-bromonicotinic acid and 1,4-diazepane for 6-methylnicotinic acid and 2-methylpyrrolidine, respectively, in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt.

### EXAMPLE 8

### (2S)-N-ethyl-1-[(6-methylpyridin-3-yl)carbonyl]pyrrolidine-2-carboxamide

The desired product was prepared by substituting L-prolinethylamide for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 262 (M+H)⁺; ¹H NMR (DMSO-d₆) δ 0.77 (t, 1H), 1.03 (t, 2H), 1.52-1.70 (m, 0.5H), 1.73-1.98 (m, 3H), 2.10-2.25 (m, 0.5H), 2.56 (s, 1H), 2.61 (s, 0.5H), 2.98-3.06 (m, 0.7H), 3.07-3.17 (m, 1.3H), 3.42-3.52 (m, 0.7H), 3.55-3.65 (m, 1.3H), 4.22 (q, 0.35H), 4.40 (q, 0.65H), 7.50 (d, 0.35H), 7.58 (d, 0.65H), 7.83-7.98 (m, 1.35H), 8.16 (dd, 0.65H), 8.57 (s, 0.35H), 8.79 (s, 0.65H).

### EXAMPLE 9

### 1-[(6-methylpyridin-3-yl)carbonyl]-4-pyridin-2-ylpiperazine

The desired product was prepared by substituting 1-(pyridin-2-yl)piperazine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 283.1 (M+H)⁺; ¹H NMR (DMSO-d₆) δ 2.58 (s, 3H), 3.47-3.80 (br m, 8H), 6.82 (t, 1H), 7.08 (d, 1H), 7.50 (d, 1H), 7.74-7.82 (m, 1H), 7.94 (dd, 1H), 8.10 (dd, 1H), 8.64 (d, 1H).

### EXAMPLE 10

### 1-(2-ethoxyphenyl)-4-[(6-methylpyridin-3-yl)carbonyl]piperazine

The desired product was prepared by substituting 1-(2-ethoxyphenyl)piperazine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 283.1 (M+H)⁺; ¹H NMR (DMSO-d₆) δ 1.45 (t, 3H), 2.86 (s, 3H), 3.45-3.55 (br m, 1H), 3.73-4.09 (br m, 5H), 4.16-4.36 (br m, 4H), 7.11-7.20 (m, 1H), 7.26 (dd, 1H), 7.49-7.59 (m, 2H), 8.03 (d, 1H); 8.58 (dd, 1H), 8.89 (d, 1H).

### EXAMPLE 11

### 2-chloro-6-methyl-3-[(2-methylpyrrolidin-1-yl)carbonyl]pyridine

The desired product was prepared by substituting 2-chloro-6-methylnicotinic acid for 6-methylnicotinic acid in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 238.9 (M+H)⁺; ¹H NMR (DMSO-d₆) δ 0.86 (d, 0.9H), 1.24 (d, 2.1H), 1.55-1.63 (m, 1H), 1.72-1.81 (m, 1H), 1.85-2.08 (m, 2H), 2.48 (s, 2H), 2.49 (s, 1H), 7.33-7.37 (m, 1H), 7.74 (d, 0.66H), 7.81 (d, 0.33H).

### EXAMPLE 12

### 2-chloro-6-methyl-3-[(2-methylpiperidin-1-yl)carbonyl]pyridine

The desired product was prepared by substituting 2-chloro-6-methylnicotinic acid and 2-methylpiperidine for 6-methylnicotinic acid and 2-methylpyrrolidine, respectively, in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 252.9 (M+H)⁺; ¹H NMR (DMSO-d₆) δ 1.10 (d, 1H), 1.20 (d, 2H), 1.32-1.75 (br m, 6H), 2.48 (d, 3H), 2.75-2.91 (br m, 0.66H), 2.99-3.12 (br m, 0.66H), 3.14-3.24 (m, 0.66H), 3.48-3.65 (br m, 0.33H), 4.34-4.42 (br m, 0.33H), 4.79-4.87 (br m, 0.33H), 7.32-7.37 (m, 1H), 7.64 (d, 0.33H), 7.72-7.78 (m, 0.66H).

### EXAMPLE 13

### 2-chloro-6-methyl-3-[(4-methylpiperidin-1-yl)carbonyl]pyridine

The desired product was prepared by substituting 2-chloro-6-methylnicotinic acid and 4-methylpiperidine for 6-methylnicotinic acid and 2-methylpyrrolidine, respectively, in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 252.9 (M+H)⁺; ¹H NMR (DMSO-d₆) δ 0.91 (d, 3H), 0.95-1.18 (br m, 2H), 1.44-1.74 (br m, 3H), 2.48 (s, 3H), 2.73-2.80 (m, 1H), 2.93-3.07 (br m, 1H), 3.19-3.26 (br m, 1H), 4.45 (br d, 1H), 7.32-7.38 (m, 1H), 7.69 (d, 0.5H), 7.76 (d, 0.5H).

### EXAMPLE 14

### 2-chloro-3-[(2-ethylpiperidin-1-yl carbonyl]-6-methylpyridine

The desired product was prepared by substituting 2-chloro-6-methylnicotinic acid and 2-ethylpiperidine for 6-methylnicotinic acid and 2-methylpyrrolidine, respectively, in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 266.9 (M+H)⁺; ¹H NMR (DMSO-d₆) δ 0.64-0.73 (m, 1H), 0.86-0.93 (m, 2H), 1.22-1.82 (br m, 8H), 2.48 (s, 3H), 2.71-2.79 (br m, 0.5H), 2.98-3.06 (br m, 1H), 3.09-3.16 (m, 0.5H), 4.35-4.46 (m, 0.5H), 4.48-4.66 (br m, 0.5H), 7.32-7.37 (m, 1H), 7.62 (d, 0.25H), 7.67 (d, 0.25H), 7.75-7.79 (m, 0.5H).

### EXAMPLE 15

### (3R)-1-[(6-methylpyridin-3-yl)carbonyl]piperidin-3-ol

The desired product was prepared by substituting (3R)-piperidin-3-ol for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 22.1.1 (M+H)⁺; ¹H NMR (DMSO-d₆) δ 1.37-1.94 (br m, 4H), 2.58 (s, 3H), 2.87 (br s, 1H), 2.98-3.14 (br m, 1H), 3.26-3.70 (br m, 3H), 4.05-4.24 (br m, 1H), 7.53 (d, 1H), 7.87 (d, 1H); 8.62 (s, 1H).

### EXAMPLE 16

### 1-[(6-methylpyridin-3-yl)carbonyl]piperidin-4-ol

The desired product was prepared by substituting piperidin-4-ol for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 221.1 (M+H)⁺; ¹H NMR (DMSO-d₆) δ 1.23-1.29 (m, 0.5H), 1.30-1.46 (br m, 1.5H), 1.75 (br d, 2H), 2.57 (s, 3H), 3.07-3.33 (br d, 2H), 3.47 (br s, 1H), 3.71-3.79 (m, 3H), 7.51 (d, 1H), 7.92 (dd, 1H), 8.59 (d, 1H).

### EXAMPLE 17

### 1-[(6-methylpyridin-3-yl)carbonyl]piperidine-3-carboxamide

The desired product was prepared by substituting nipecotamide for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 248.1 (M+H)⁺; ¹H NMR (DMSO-d₆) δ 1.40-1.78 (br m, 3H), 1.88-1.98 (br m, 1H), 2.33-2.44 (br m, 1H), 2.77 (s, 3H), 2.83-2.95 (br m, 0.5H), 3.03-3.13 (m, 1H), 3.27 (br t, 0.5H), 3.47 (br d, 1H), 4.09 (br d, 0.5H), 4.43 (br d, 0.5H), 6.88 (br d, 1H), 7.44 (br d, 1H), 7.90 (d, 1H), 8.33-8.46 (br m, 1H), 8.88 (br s, 1H).

### Alternative Procedure for the Preparation of Example 17

A stirred solution of 6-methylnicotinic acid (8 mmol) in DMF (15 mL) was treated with N-hydroxysuccinimide (9.5 mmol). While the mixture was stirred at room temperature a solution formed. The solution was treated with 1,3-dicyclohexylcarbodiimide (8.8 mmol), stirred for 2.5 hours, treated with glacial acetic acid (0.14 mL), stirred for 30 minutes, and then filtered. The filtrate was concentrated under vacuum and the residue was dissolved in hot ethyl acetate. The solution was filtered while hot and the filtrate was cooled to room temperature which resulted in the formation of a precipitate. The precipitate was collected by filtration to provide the N-hydroxysuccinimide ester of 6-methylnicotinic acid. MS m/e 235 (M+H)⁺; ¹H NMR (DMSO-d₆) δ 8.96 (d, 1H), 8.20 (dd, 1H), 7.42 (d, 1H), 2.77 (s, 4H), 2.49 (s, 3H).

A solution of the above ester (1 mmol) and nipecotamide (1.19 mmol) in dichloromethane (8 mL) was stirred at room temperature overnight and then heated to reflux for 1 hour. The mixture was cooled to room temperature, washed three times with sodium bicarbonate, water and brine, dried (Na₂SO₄), filtered, and concentrated. The residue was crystallized from ethyl acetate to provide the desired product.

### EXAMPLE 18

### 1-[(6-methylpyridin-3-yl)carbonyl]piperidine-4-carboxamide

The desired product was prepared by substituting isonipecotamide for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 248.1 (M+H)⁺; ¹H NMR (DMSO-d₆) δ 1.45-1.58 (m, 2H), 1.74 (br d, 2H), 2.34-2.42 (m, 1H), 2.57 (s, 3H), 2.86 (br s, 1H), 3.03-3.19 (br m, 1H), 3.56 (br s, 1H), 4.41 (br s, 1H), 6.89 (br s, 1H), 7.27 (br s, 1H), 7.51 (d, 1H), 7.92 (dd, 1H), 8.59 (d, 1H).

### EXAMPLE 19

### N,N-diethyl-1-[(6-methylpyridin-3-yl)carbonyl]piperidine-3-carboxamide

The desired product was prepared by substituting N,N-diethylnipecotamide for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 304.2 (M+H)⁺; ¹H NMR (DMSO-d₆) δ 0.85-1.21 (br m, 6H), 1.44-1.86 (br m, 4H), 2.56 (s, 3H), 2.70-2.78 (m, 1H), 2.80-2.91 (m, 1H), 3.00-3.15 (br m, 1H), 3.22-3.45 (br m, 4H), 3.51 (br d, 1H), 4.37 (br t, 1H), 7.50 (d, 1H), 7.93 (d, 1H), 8.60 (d, 1H).

### EXAMPLE 20

### 5-[(4-benzylpiperidin-1-yl)carbonyl]-2-methylpyridine

The desired product was prepared by substituting 4-benzylpiperidine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 295.1 (M+H)⁺; ¹H NMR (DMSO-d₆) δ 1.09-1.22 (m, 2H), 1.45-1.71 (br m, 2H), 1.74-1.84 (m, 1H), 2.52 (d, 2H), 2.56 (s, 3H), 2.65-2.82 (br m, 1H), 2.93-3.07 (br m, 1H), 3.51 (br s, 1H), 4.43 (br s, 1H), 7.14-7.22 (m, 3H), 7.24-7.32 (m, 2H), 7.50 (d, 1H), 7.91 (dd, 1H), 8.58 (d, 1H).

### EXAMPLE 21

### 1-{1-[(6-methylpyridin-3-yl)carbonyl]piperidin-4-yl}-1,3-dihydro-2H-benzimidazol-2-one

The desired product was prepared by substituting 1-piperidin-4-yl-1,3-dihydro-2H-benzimidazol-2-one for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 337.2 (M+H)⁺; ¹H NMR (DMSO-d₆) δ 1.75 (br d, 2H), 2.25-2.39 (br m, 2H), 2.60 (s, 3H), 2.88-3.05 (br m, 1H), 3.19-3.37 (br m, 1H), 3.59-3.76 (br m, 1H), 4.44-4.53 (m, 2H), 6.96-7.39 (m, 3H), 7.35-7.39 (m, 1H), 7.58 (d, 1H), 8.07 (dd, 1H), 8.72 (d, 1H), 10.85 (s, 1H).

### EXAMPLE 22

### 1-methyl-4-[(6-methylpyridin-3-yl)carbonyl]piperazine

The desired product was prepared by substituting 1-(methyl)piperazine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 220.1 (M+H)⁺; ¹H NMR (DMSO-d₆) δ 2.53 (s, 3H), 2.77 (br s, 2H), 2.82 (s, 3H), 3.07 (br t, 2H), 3.29 (br t, 4H), 7.39 (d, 1H), 7.79 (dd, 1H), 8.52-8.56 (m, 1H).

### EXAMPLE 23

### 4-[(6-methylpyridin-3-yl)carbonyl]piperazine-1-carbaldehyde

The desired product was prepared by substituting 1-piperazinecarboxaldehyde for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 234.1 (M+H)⁺; ¹H NMR (DMSO-d₆) δ 2.53-2.58 (m, 3H), 3.17 (br s, 2H), 3.44 (br s, 4H), 3.66 (br s, 2H), 7.47 (q, 1H), 7.81-7.95 (m, 1H), 8.07 (s, 0.7514), 8.14 (s, 0.25H), 8.61 (s, 1H).

### EXAMPLE 24

### 1-benzyl-4-[(6-methylpyridin-3-yl)carbonyl]piperazine

The desired product was prepared by substituting 1-(benzyl)piperazine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 296.1 (M+H)⁺; ¹H NMR (DMSO-d₆) δ 2.55 (s, 3H), 3.02-3.52 (br m, 6H), 4.35 (s, 2H), 7.40-7.53 (m, 6H), 7.86 (dd, 1H), 8.58 (dd, 1H).

### EXAMPLE 25

### 1-(4-fluorophenyl)-4-[(6-methylpyridin-3-yl)carbonyl]piperazine

The desired product was prepared by substituting 1-(4-fluorophenyl)piperazine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 300.1 (M+H)⁺; ¹H NMR (DMSO-d₆) δ 2.57 (s, 3H), 3.13 (br s, 4H), 3.50 (br s, 2H), 3.78 (br s, 2H), 6.96-7.01 (m, 2H), 7.04-7.12 (m, 2H), 7.51 (d, 1H), 7.95 (dd, 1H), 8.63 (d, 1H).

### EXAMPLE 26

### 1-methyl-4-[(6-methylpyridin-3-yl)carbonyl]-1,4-diazepane

The desired product was prepared by substituting 1-methyl-1,4-diazepane for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 234.1 (M+H)⁺; ¹H NMR (DMSO-d₆) δ 1.97-2.19 (br m, 2H), 2.53 (s, 3H), 2.80-2.91 (br m, 3H), 3.17-3.61 (br m, 7H), 4.04-4.17 (br m, 1H), 7.41 (d, 1H), 7.82 (dd, 1H), 8.57 (s, 1H).

### EXAMPLE 27

### 5-[(2,5-dimethylpyrrolidin-1-yl)carbonyl]-2-methylpyridine

The desired product was prepared by substituting 2,5-dimethylpyrrolidine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 219 (M+H)⁺; ¹H NMR (DMSO-d₆) δ 0.48 (d, 0.5H), 0.56-1.17 (br m, 5.5H), 1.22-1.50 (br m, 2H), 1.59-2.05 (br m, 2H), 2.91 (s, 3H), 3.40-4.04 (br m, 2H), 7.63 (d, 1H), 8.17 (dd, 0.65H), 8.22 (dd, 0.15H), 8.58 (d, 0.65H), 8.67 (d, 0.15H).

### EXAMPLE 28

### {(2S)-1-[(6-methylpyridin-3-yl)carbonyl]pyrrolidin-2-yl}methanol

The desired product was prepared by substituting (2S)-2-pyrrolidinylmethanol for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 221.1 (M+H)⁺; ¹H NMR (DMSO-d₆) δ 1.60-2.02 (br m, 4H), 2.56 (s, 3H), 3.01-3.16 (br m, 0.5H), 3.25-3.38 (br m, 1H), 3.38-3.65 (m, 3H), 3.78-3.91 (br s, 0.5H), 4.09-4.19 (br m, 1H), 7.47 (d, 1H), 7.99 (dd, 1H), 8.67 (d, 1H).

### EXAMPLE 29

### {(2R)-1-[(6-methylpyridin-3-yl)carbonyl]pyrrolidin-2-yl}methanol

The desired product was prepared by substituting (2R)-2-pyrrolidinylmethanol for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 221.1 (M+H)⁺; ¹H NMR (DMSO-d₆) δ 1.62-2.02 (br m, 4H), 2.55 (s, 3H), 3.02-3.15 (br m, 0.5H), 3.24-3.38 (br m, 1H), 3.39-3.67 (m, 3H), 3.77-3.91 (br s, 0.5H), 4.08-4.21 (br m, 1H), 7.44 (d, 1H), 7.95 (dd, 1H), 8.64 (d, 1H).

### EXAMPLE 30

### 3-bromo-5-[(2-methylpyrrolidin-1-yl)carbonyl]pyridine

The desired product was prepared by substituting 5-bromonicotinic acid for 6-methylnicotinic acid in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 269.0 (M+H)⁺; ¹H NMR (DMSO-d₆) δ 0.86 (d, 0.75H), 1.25 (d, 2.25H), 1.50-1.63 (m, 1H), 1.66-1.80 (m, 1H), 1.81-1.96 (m, 1H), 2.02-2.12 (m, 1H), 3.28-3.35 (m, 0.5H), 3.46-3.55 (m, 1.5H), 3.88-3.98 (m, 0.25H), 4.10-4.20 (m, 0.75H), 8.15-8.22 (m, 1H), 8.64-8.69 (m, 1H), 8.78 (d, 1H).

### EXAMPLE 31

### 2-bromo-5-[(2-methylpyrrolidin-1-yl)carbonyl]pyridine

The desired product was prepared by substituting 6-bromonicotinic acid for 6-methylnicotinic acid in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 268.9 (M+H)⁺; ¹H NMR (DMSO-d₆) δ 0.86 (d, 0.75H), 1.25 (d, 2.25H), 1.48-1.63 (m, 1H), 1.66-1.80 (m, 1H), 1.81-1.97 (m, 1H), 2.00-2.13 (m, 1H), 3.27-3.37 (m, 0.5H), 3.45-3.54 (m, 1.5H), 3.88-4.00 (m, 0.25H), 4.09-4.21 (m, 0.75H), 7.72 (d, 1H), 7.87 (dd, 1H), 8.52 (d, 1H).

### EXAMPLE 32

### 2-methyl-5-{[(2R)-2-methylpyrrolidin-1-yl]carbonyl}pyridine

A suspension of N-cyclohexylcarbodiimide-N-methylpolystyrene HL resin (purchased from Novabiochem Corp., substitution 1.69 mmol/g, 1.2 g) in dichloromethane (10 mL) was gently shaken for 30 minutes. The mixture was treated with a solution of 6-methylnicotinic acid (0.137 g, 1.0 mmol), 1-hydroxy-7-azabenzotriazole (0.1361 g, 1.0 mmol) and diisopropylamine (0.5 mL, 3.0 mmol) in DMF (5.0 mL), gently shaken for ten minutes, treated with (2R)-2-methylpyrrolidine tartarate salt (0.2235 g, 0.95 mmol), shaken overnight, and filtered. The resin was washed three times with dichloromethane. The filtrate and the washes were combined, treated with PS-trisamine resin (purchased from Argonaut Technologies, substitution 4.42 mmol/g, 0.5 g), and gently shaken for two hours. The suspension was filtered and the resin was washed with dichloromethane. The filtrate and the washes were concentrated and the concentrate was purified by HPLC on a C-18 column using a solvent system varying in a gradient of 10% to 50% acetonitrile/water containing 0.1% TFA. The combined fractions were lyophilized to provide the desired product as the trifluoroacetate salt (0.255 g). The salt was dissolved in dichloromethane, treated with PS-trisamine (0.5 g) for ten minutes, and filtered. The filtrate was concentrated and dissolved in diethyl ether. The solution was treated with 2 M HCl in diethyl ether (2 mL) and filtered. The filter cake was recrystallized from methanol/ethyl acetate/hexane to provide the desired product as the hydrochloride salt (0.148 g). MS m/e 205:1 (M+H)⁺; ¹H NMR (DMSO-d₆) δ 0.85 (d, 0.7H), 1.25 (d, 2.30H), 1.49-1.63 (m, 1H), 1.65-1.79 (m, 1H), 1.81-1.90 (m, 1H), 2.01-2.10 (m, 1H), 2.76 (s, 3H), 3.29-3.39 (m, 0.7H), 3.46-3.57 (m, 1.3H), 3.95-4.0 (m, 0.25H), 4.09-4.20 (m, 0.75H), 7.40 (dd, 1H), 8.48 (dd, 1H), 8.82-8.92 (m, 1H).

### EXAMPLE 33

### 2-methyl-5-{[(2S)-2-methylpyrrolidin-1-yl]carbonyl}pyridine

The desired product was prepared by substituting (2S)-2-methylpyrrolidine for 2-methylpyrrolidine in Example 1. After workup the crude compound was purified by HPLC on a C-18 column using a solvent system increasing over 50 minutes in a gradient of 5% to 100% acetonitrile/water containing 0.01% TFA to provide the desired product as the trifluoroacetate salt. MS m/e 205.1 (M+H)⁺; ¹H NMR (DMSO-d₆) δ 0.87 (d, 0.65H), 1.27 (d, 2.35H), 1.50-1.65 (m, 1H), 1.66-1.82 (m, 1H), 1.82-2.00 (m, 1H), 2.02-2.15 (m, 1H), 2.76 (s, 3H), 3.30-3.40 (m, 0.6H), 3.46-3.59 (m, 1.4H), 3.92-4.02 (m, 0.30H), 4.11-4.21 (m, 0.7H), 7.88 (d, 1H), 8.47 (dd, 1H), 8.84-8.92 (m, 1H).

### EXAMPLE 34

### 2-methyl-3-[(2-methyl-1-pyrrolidinyl)carbonyl]pyridine

The desired product was prepared by substituting 2-methylnicotinic acid for 6-methylnicotinic acid in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 205.1 (+H)⁺; ¹H NMR (DMSO-d₆) δ 0.84 (d, 1H), 1.28 (d, 2H), 1.53-1.66 (m, 1H), 1.69-2.15 (m, 3H), 2.60 (s, 1H), 2.64 (s, 2H), 3.07-3.28 (m, 1.4H), 3.52-3.62 (m, 0.6H), 3.66-3.76 (m, 0.35H), 4.14-4.27 (m, 0.65H), 7.77-7.86 (m, 1H), 8.33-8.40 (m, 1H), 8.73-8.80 (m, 1H).

### EXAMPLE 35

### 4-methyl-3-[(2-methyl-1-pyrrolidinyl)carbonyl]pyridine

The desired product was prepared by substituting 4-methylnicotinic acid for 6-methylnicotinic acid in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 205.1 (M+H)⁺; ¹H NMR (DMSO-d₆) δ 0.83 (d, 1H), 1.28 (d, 2H), 1.54-1.66 (m, 1H), 1.69-2.14 (m, 3H), 2.43 (s, 1H), 2.47 (s, 2H), 3.07-3.25 (m, 1.4H), 3.48-3.62 (m, 0.6H), 3.65-3.75 (m, 0.35H), 4.15-4.27 (m, 0.65H), 7.84-7.91 (m, 1H), 8.76 (d, 1H), 8.83 (s, 0.7H), 8.90 (s, 0.3H).

### EXAMPLE 36

### 3-methyl-5-[(2-methyl-1-pyrrolidinyl)carbonyl]pyridine

The desired product was prepared by substituting 5-methylnicotinic acid for 6-methylnicotinic acid in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 205.1 (M+H)⁺; ¹H NMR (DMSO-d₆) δ 0.86 (d, 0.8H), 1.27 (d, 2.2H), 1.50-2.16 (m, 4H), 2.47 (s, 3H), 3.27-3.40 (m, 0.75H), 3.45-3.59 (m, 1.25H), 3.90-4.02 (m, 0.25H), 4.09-4.24 (m, 0.75H), 8.25-8.36 (m, 1H), 8.76 (s, 1H), 8.80 (d, 1H).

### EXAMPLE 37

### 5-{[(2S)-2-(methoxymethyl)-1-pyrrolidinyl]carbonyl}-2-methylpyridine

The desired product was prepared by substituting (2S)-2-(methoxymethyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 235.1 (M+H)⁺; ¹H NMR (DMSO-d₆) δ 1.62-2.08 (br m, 4H), 2.71 (s, 3H), 2.97-3.14 (br m, 1.25H), 3.30 (s, 3H), 3.31-3.52 (m, 2H), 3.54-3.68 (br m, 0.75H), 4.01 (br s, 0.25H), 4.26 (br s, 0.75H), 7.79 (d, 1H), 8.35 (d, 1H), 8.83 (s, 1H).

### EXAMPLE 38

### 2-methyl-5-{[(2S)-2-(1-pyrrolidinylmethyl)-1-pyrrolidinyl]carbonyl}pyridine

The desired product was prepared by substituting 1-[(2S)-2-pyrrolidinylmethyl]pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. The precipitate was isolated by filtration to provide the desired product as the dihydrochloride salt. MS m/e 274.1 (M+H)⁺; ¹H NMR (DMSO-d₆) δ 1.75-2.17 (br m, 8H), 2.75 (s, 3H), 2.97-3.29 (m, 3H), 3.30-3.49 (m, 2H), 3.52-3.83 (m, 3H), 4.54-4.65 (m, 1H), 7.87 (d, 1H), 8.55 (dd, 1H), 9.05 (d, 1H), 10.64 (br s, 1H).

### EXAMPLE 39

### benzyl (2S)-1-[(6-methyl-3-pyridinyl)carbonyl]-2-pyrrolidinecarboxylate

The desired product was prepared by substituting benzyl (2S)-2-pyrrolidinecarboxylate for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 325.1 (M+H)⁺; ¹H NMR (DMSO-d₆) δ 1.74-2.03 (m, 3H), 2.23-2.41 (m, 1H), 2.61 (s, 0.6H), 2.67 (s, 2.4H), 3.50-3.68 (m, 2H), 4.52-4.61 (m, 1H), 4.62-4.71 (m, 0.5H), 5.18 (d, 1.5H), 7.12-7.22 (m, 0.4H), 7.30-7.47 (m, 4.6H), 7.58 (d, 0.2H), 7.72 (d, 0.8H), 8.05 (dd, 0.2H), 8.27 (dd, 0.8H), 8.71 (d, 0.2H), 8.80 (d, 0.8H).

### EXAMPLE 40

### 5-{[(2R,5R)-2,5-bis(methoxymethyl)-1-pyrrolidinyl]carbonyl}-2-methylpyridine

The desired product was prepared by substituting (2R,5R)-2,5-bis(methoxymethyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 279.1 (M+H)⁺; ¹H NMR (DMSO-d₆) δ 1.67-1.90 (m, 2H), 1.93-2.27 (m, 2H), 2.71 (s, 3H), 2.87-3.06 (m, 5H), 3.29 (s, 3H), 3.31-3.40 (m, 1H), 3.47-3.58 (m, 1H), 4.11 (br q, 1H), 4.24-4.34 (br m, 1H), 7.77 (d, 1H), 8.32 (dd, 1H), 8.84 (d, 1H).

### EXAMPLE 41

### 5-{[(2S,5S)-2,5-bis(methoxymethyl)-1-pyrrolidinyl]carbonyl}-2-methylpyridine

The desired product was prepared by substituting (2S,5S)-2,5-bis(methoxymethyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 279.1 (M+H)⁺; ¹H NMR (DMSO-d₆) δ 1.67-1.91 (m, 2H), 1.93-2.29 (m, 2H), 2.71 (s, 3H), 2.86-3.06 (m, 5H), 3.20-341 (m, 4H), 3.46-3.59 (m, 1H), 4.11 (br q, 1H), 4.22-4.35 (br m, 1H), 7.78 (d, 1H), 8.33 (dd, 1H), 8.84 (d, 1H).

### EXAMPLE 42

### 5-[(2-isopropyl-1-pyrrolidinyl)carbonyl]-2-methylpyridine

The desired product was prepared by substituting 2-isopropylpyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 233.1 (M+H)⁺; ¹H NMR (DMSO-d₆) δ 0.89 (t, 6H), 1.59-1.95 (m, 4H), 2.23-2.37 (m, 1H), 2.71 (s, 3H), 3.29-3.53 (m, 2H), 4.09 (q, 1H), 7.79 (d, 1H), 8.38 (dd, 1H), 8.84 (d, 1H).

### EXAMPLE 43

### 2-methyl-5-{[2-(3-pyridinyl)-1-pyrrolidinyl]carbonyl}pyridine

The desired product was prepared by substituting 3-(2-pyrrolidinyl)pyridine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 268.1 (M+H)⁺; ¹H NMR (DMSO-d₆) δ 1.77-2.04 (m, 4H), 2.71 (s, 3H), 3.53-3.65 (m, 1H), 3.90-4.03 (m, 1H), 5.28 (t, 1H), 7.77 (d, 1H), 8.03 (q, 1H), 8.41 (dd, 1H), 8.65-8.71 (m, 1H), 8.81 (d, 1H), 9.00 (d, 1H), 9.09 (d, 1H).

### EXAMPLE 44

### 2-methyl-5-{[2-(2-phenylethyl)-1-pyrrolidinyl]carbonyl}pyridine

The desired product was prepared by substituting 2-(2-phenylethyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 295.1 (M+H)⁺; ¹H NMR (DMSO-d₆) δ 1.58-2.32 (m, 6H), 2.54-2.78 (m, 5H), 3.27-3.42 (m, 0.75H), 3.43-3.60 (m, 1.25H), 3.66 (br s, 0.2H), 4.09-4.23 (br m, 0.8H), 6.83-6.93 (br m, 0.5H), 7.09-7.33 (m, 4.5H), 7.67 (d, 0.25H), 7.80 (d, 0.75H), 8.25 (dd, 0.25H), 8.35 (dd, 0.75H), 8.75-8.85 (m, 1H).

### EXAMPLE 45

### 2-methyl-5-[(2-phenyl-1-pyrrolidinyl)carbonyl]pyridine

The desired product was prepared by substituting 2-(phenyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 267.1 (M+H)⁺; ¹H NMR (DMSO-d₆) δ 1.67-2.01 (m, 3H), 2.31-2.46 (m, 1H), 2.57 (s, 1H), 2.72 (s, 2H), 3.49-3.61 (m 0.75H), 3.74-3.92 (m, 1.25H), 4.93-5.01 (br m, 0.3H), 5.16 (t, 0.7H), 7.00 (d, 0.6H), 7.12-7.27 (m, 1.7H), 7.29-7.42 (m, 2.7H), 7.51 (d, 0.35H), 7.81 (d, 0.65H), 7.90 (dd, 0.35H), 8.42-8.54 (m, 1H), 8.95 (d, 0.65H).

### EXAMPLE 46

### N-{(3R)-1-[(6-methyl-3-pyridinyl)carbonyl]-3-pyrrolidinyl}acetamide

The desired product was prepared by substituting N-[(3R)-3-pyrrolidinyl]acetamide for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 248.1 (M+H)⁺; ¹H NMR (DMSO-d₆) δ 1.71-1.97 (m, 4H), 1.99-2.17 (m, 1H), 2.74 (d, 3H), 3.22 (dd, 0.7H), 3.30-3.74 (m, 3.3H), 4.13-4.37 (m, 1H), 7.88 (dd, 1H), 8.24 (d, 0.55H), 8.31 (d, 0.45H), 8.41-8.51 (m, 1H), 8.90 (dd, 1H).

### EXAMPLE 47

### N-{(3S)-1-[(6-methyl-3-pyridinyl)carbonyl]-3-pyrrolidinyl}acetamide

The desired product was prepared by substituting N-[(3S)-3-pyrrolidinyl]acetamide for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 248.1 (M+H)⁺; ¹H NMR (DMSO-d₆) δ 1.70-1.94 (m, 4H), 1.97-2.17 (m, 1H), 2.73 (d, 3H), 3.22 (dd, 0.7H), 3.29-3.74 (m, 3.3H), 4.13-4.37 (m, 1H), 7.86 (dd, 1H), 8.24 (d, 0.55H), 8.32 (d, 0.45H), 8.40-8.50 (m, 1H), 8.90 (dd, 1H).

### EXAMPLE 48

### (3R)-1-[(6-methyl-3-pyridinyl)carbonyl]-3-pyrrolidinamine

The desired product was prepared by substituting tert-butyl (3R)-3-pyrrolidinylcarbamate for 2-methylpyrrolidine in Example 1 (downsized to a 1 mmol scale). After workup the crude compound was treated with a mixture of TFA/dichloromethane (1:1) for 1 hour and concentrated. The concentrate was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 206.0 (M+H)⁺; ¹H NMR (DMSO-d₆) δ 1.90-2.08 (br m, 1H), 2.14-2.32 (m, 1H), 2.55 (s, 3H), 3.39-3.93 (m, 5H), 7.43 (d, 1H), 7.86-7.96 (m, 1H), 8.09 (br d, 3H), 8.65 (d, 1H).

### EXAMPLE 49

### (3S)-1-[(6-methyl-3-pyridinyl)carbonyl]-3-pyrrolidinamine

The desired product was prepared by substituting tert-butyl (3S)-3-pyrrolidinylcarbamate for 2-methylpyrrolidine in Example 1 (downsized to a 1 mmol scale). After workup the crude compound was treated with a mixture of TFA/dichloromethane (1:1) for 1 hour and concentrated. The concentrate was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 206.0 (M+H)⁺; ¹H NMR (DMSO-d₆) δ 1.92-2.09 (br m, 1H), 2.15-2.32 (m, 1H), 2.55 (s, 3H), 3.39-3.95 (m, 5H), 7.45 (d, 1H), 7.88-7.99 (m, 1H), 8.13 (br d, 3H), 8.66 (d, 1H).

### EXAMPLE 50

### (3S)-N,N-dimethyl-1-[(6-methyl-3-pyridinyl)carbonyl]-3-pyrrolidinamine

The desired product was prepared by substituting (3S)-N,N-dimethyl-3-pyrrolidinamine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 234.1 (M+H)⁺; ¹H NMR (DMSO-d₆) δ 2.03-2.19 (m, 1H), 2.24-2.41 (br m, 1H), 2.53 (s, 3H), 2.68-2.93 (br m, 6H), 3.48-4.00 (m, 5H), 7.38 (d, 1H), 7.87 (dd, 1H), 8.63 (d, 1H).

### EXAMPLE 51

### (3R)-N,N-dimethyl-1-[(6-methyl-3-pyridinyl)carbonyl]-3-pyrrolidinamine

The desired product was prepared by substituting (3R)-N,N-dimethyl-3-pyrrolidinamine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 234.1 (M+H)⁺; ¹H NMR (DMSO-d₆) δ 2.04-2.19 (m, 1H), 2.26-2.42 (br m, 1H), 2.53 (s, 3H), 2.70-2.95 (br m, 6H), 3.47-3.99 (br m, 5H), 7.39 (d, 1H), 7.89 (dd, 1H), 8.64 (d, 1H).

### EXAMPLE 52

### 1-{[5-(2,5-dimethylphenyl)-3-pyridinyl]carbonyl}-3-piperidinecarboxamide

The desired product was prepared by substituting nipecotamide for 2-methylpyrrolidine in Example 59. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 338.1 (M+H)⁺; ¹H NMR (DMSO-d₆) δ 1.37-1.82 (br m, 3H), 1.92 (br s, 1H), 2.21 (s, 3H), 2.30-2.43 (m, 4H), 2.77-3.33 (br m, 2H), 3.54 (br s, 1H), 4.26 (br s, 1H), 6.79-6.97 (br m, 1H), 7.10-7.27 (m, 3H), 7.35 (br d, 1H), 7.90 (br s, 1H), 8.64 (s, 1H), 8.68 (d, 1H).

### EXAMPLE 53

### 2-methyl-5-[(3-phenyl-1-pyrrolidinyl)carbonyl]pyridine

The desired product was prepared by substituting 3-phenylpyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 267.0 (M+H)⁺; ¹H NMR (DMSO-d₆) δ 1.93-2.12 (m, 1H), 2.23-2.38 (m, 1H), 2.71-2.81 (m, 3H), 3.35-3.71 (m, 3.5H), 3.72-3.87 (m, 1H), 3.95-4.07 (m, 0.5H), 7.20-7.39 (m, 5H), 7.89 (t, 1H), 8.51 (dd, 1H), 8.88-8.93 (m, 1H).

### EXAMPLE 54

### 5-[(3-benzyl-1-pyrrolidinyl)carbonyl]-2-methylpyridine

The desired product was prepared by substituting 3-benzylpyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 281.1 (M+H)⁺; ¹H NMR (DMSO-d₆) δ 1.93-2.12 (m, 1H), 1.52-1.73 (m, 1H), 1.83-2.03 (m, 1H), 2.57-2.80 (m 5H), 3.12-3.26 (m, 1H), 3.36-3.70 (m, 4H), 7.12-7.38 (m, 5H), 7.76 (t, 1H), 8.29-8.39 (m, 1H), 8.84 (dd, 1H).

### EXAMPLE 55

### 2-methyl-5-{[3-(2-phenylethyl)-1-pyrrolidinyl]carbonyl}pyridine

The desired product was prepared by substituting 3-(2-phenylethyl)pyrrolidine for 2-methylpyrrolidine in Example 1 (downsized to a 1 mmol scale). After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 295.1 (M+H)⁺; ¹H NMR (DMSO-d₆) δ 1.48-1.80 (m, 3H), 1.95-2.25 (m, 2H), 2.51-2.70 (m, 2H), 2.71-2.79 (m, 3H), 3.07-3.19 (m, 1H), 3.35-3.77 (m, 3H), 7.11-7.34 (m, 5H), 7.89 (dd, 1H), 8.44-8.53 (m, 1H), 8.89 (dd, 1H).

### EXAMPLE 56

### (3R)-1-[(6-methyl-3-pyridinyl)carbonyl]-3-piperidinecarboxamide

In the reaction vessel of a Rainin Symphony peptide synthesizer was added 0.2 mmol (substitution 0.72 mmol/g) of Fmoc-Rink amide MBHA resin. Using the following synthetic protocol (R)-Fmoc-nipecotic acid and 6-methylnicotinic acid were sequentially coupled to the resin:
1. resin solvated three times for 15 minutes with DMF;
2. deprotected twice with 20% piperidine for 15 minutes;
3. washed six times with DMF;
4. resin treated with 3.75 mL of 0.3M (R)-Fmoc-nipecotic acid (11.25 mmol) in DMF;
5. coupled to the above carboxylic acid by treating the suspension of step 4 with a 0.3M solution of HBTU in DMF containing a 0.4M solution of N-methylmorpholine in DMF (3.75 mL) and then shaking for 20 minutes;
6. resin washed three times with DMF;
7. steps 2-6 repeated for 6-methylnicotinic acid coupling;
8. product cleaved from the resin upon treatment with a cocktail solution of 95% TFA/2.5% H₂O/2.5% anisole (5 mL) for 3 hours.

Upon completion of the cleavage, removal of the resin by filtration, and concentration in vacuo of the filtrate, the residue was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 248.0 (M+H)⁺; ¹H NMR (DMSO-d₆) δ 1.37-1.79 (br m, 3H), 1.85-2.00 (m, 1H), 2.30-2.43 (m, 1H), 2.74 (s, 1H), 2.81-2.97 (br m, 0.5H), 3.00-3.13 (m, 1H), 3.18-3.32 (m, 0.5H), 3.38-3.53 (br m, 1H), 4.10 (br d, 0.5H), 4.43 (br d, 0.5H), 6.87 (br d, 1H), 7.41 (br d, 1H), 7.86 (d, 1H), 8.26-8.43 (br m, 1H), 8.79 (br s, 1H).

### EXAMPLE 57

### (3S)-1-[(6-methyl-3-pyridinyl)carbonyl]-3-piperidinecarboxamide

The desired product was prepared by substituting (S)-Fmoc-nipecotic acid for (R)-Fmoc-nipecotic acid in Example 56. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. The salt was dissolved in dichloromethane (10 mL) and shaken with basic resin MP carbonate (0.75 g) for four hours. The resin was removed by filtration and the filtrate was concentrated in vacuo. The residue was dissolved in diethyl ether (10 mL) and treated dropwise with 1M HCl in diethyl ether (5 mL). The precipitate was isolated by filtration to provide the desired product as the hydrochloride salt. MS m/e 248.0 (M+H)⁺; ¹H NMR (DMSO-d₆) δ 1.38-1.79 (br m, 3H), 1.87-1.99 (m, 1H), 2.33-2.45 (br m, 1H), 2.77 (s, 1H), 2.82-2.97 (br m, 0.5H), 3.01-3.14 (m, 1H), 3.19-3.34 (m, 0.5H), 3.40-3.54 (br m, 1H), 4.09 (br d, 0.5H), 4.43 (br d, 0.5H), 6.88 (br d, 1H), 7.44 (br d, 1H), 7.91 (d, 1H), 8.34-8.49 (br m, 1H), 8.81 (br s, 1H).

### EXAMPLE 58

### 3-[(2-methylpyrrolidin-1-yl)carbonyl]-5-phenylpyridine

A solution of the compound described in Example 30 (1 mmol), phenylboronic acid (2.0 mmol), and tetrakis(triphenylphosphine)palladium (0) (0.05 mmol) in dichloromethane (1.5 mL) and ethanol (0.25 mL) was treated with 2 M sodium carbonate (0.5 mL), heated to 87° C. overnight, and concentrated. The residue was dissolved in diethyl ether, washed three times with water, dried (Na₂SO₄), filtered and concentrated. The concentrate was purified by HPLC using a C-18 column with a solvent system increasing in gradient over 50 minutes from 5% to 100% acetonitrile/water containing 0.01% TFA and lyophilized to provide the desired product as the trifluoroacetate salt. MS m/e 267.1 (M+H)⁺; ¹H NMR (DMSO-d₆) δ 0.88 (d, 0.8H), 1.27 (d, 2.2H), 1.53-1.62 (m, 1H), 1.69-1.79 (m, 1H), 1.85-1.97 (m, 1H), 2.04-2.14 (m, 1H), 3.34-3.41 (m, 0.6H), 3.51-3.62 (m, 1.4H), 3.96-4.06 (m, 0.25H), 4.15-4.24 (m, 0.75H), 7.43-7.55 (m, 3H), 7.79 (d, 2H), 8.15 (s, 1H), 8.62-8.69 (m, 1H), 8.93-9.99 (m, 1H).

### EXAMPLE 59

### 3-(2,5-dimethylphenyl)-5-[(2-methylpyrrolidin-1-yl)carbonyl]pyridine

A solution of the compound described in Example 30, 2,5-dimethylphenylboronic acid (2.0 mmol) and tetrakis(triphenylphosphine)palladium (0.05 mmol) in dichloromethane (1.5 mL) and ethanol (0.25 mL) was treated with 2 M sodium carbonate (0.5 mL), heated to 87° C. overnight, and concentrated. The residue was dissolved in diethyl ether, washed with water three times, dried (Na₂SO₄), filtered, and concentrated. The concentrate was purified by HPLC using a C-18 column with a solvent system increasing in gradient over 50 minutes from 5% to 100% acetonitrile/water containing 0.01% TFA and lyophilized to provide the desired product. MS m/e 295 (M+H)⁺; ¹H NMR (DMSO-d₆) δ 0.88 (d, 0.75H), 1.27 (d, 2.25H), 1.50-1.63 (m, 1H), 1.68-1.80 (m, 1H), 1.84-1.98 (m, 1H), 2.04-2.13 (m, 1H), 2.20 (s, 3H), 2.32 (s, 3H), 3.34-3.44 (m, 0.75H), 3.49-3.60 (m, 1.25H), 4.01 (br s, 0.25H), 4.14-4.23 (m, 0.75H), 7.10 (s, 1H), 7.15 (dd, 1H), 7.23 (d, 1H), 7.84 (t, 1H), 8.10 (d, 1H), 8.62-8.69 (m, 1H).

### EXAMPLE 60

### 3-(4-methoxyphenyl)-5-[(2-methylpyrrolidin-1-yl)carbonyl]pyridine

A solution of the compound described in Example 30, 4-methoxyphenylboronic acid (2.0 mmol), and tetrakis(triphenylphosphine)palladium (0.05 mmol) in dichloromethane (1.5 mL) and ethanol (0.25 mL) was treated with 2 M sodium carbonate (0.5 mL), heated to 87° C. overnight, and concentrated. The residue was dissolved in diethyl ether, washed with water three times, dried (Na₂SO₄), filtered, and concentrated. The concentrate was purified by HPLC using a C-18 column with a solvent system increasing in gradient over 50 minutes from 5% to 100% acetonitrile/water containing 0.01% TFA and lyophilized to provide the desired product as the trifluoroacetate salt. MS m/e 297 (M+H)⁺; ¹H NMR (DMSO-d₆) δ 0.87 (d, 0.75H), 1.28 (d, 2.25H), 1.52-1.62 (m, 1H), 1.67-1.79 (m, 1H), 1.84-1.98 (m, 1H), 2.03-2.14 (m, 1H), 3.33-3.41 (m, 0.75H), 3.50-3.61 (m, 1.25H), 3.82 (s, 3H), 4.00 (br s, 0.25H), 4.14-4.24 (m, 0.75H), 7.07 (d, 2H), 7.74 (d, 2H), 8.09 (s, 1H), 8.54-8.62 (m, 1H), 8.92 (d, 1H).

### EXAMPLE 61

### 3-(3-chlorophenyl)-5-[(2-methylpyrrolidin-1-yl)carbonyl]pyridine

A solution of the compound described in Example 30 (1 mmol), (3-chloro)phenylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 62

### 3-{5-[(2-methylpyrrolidin-1-yl)carbonyl]pyridin-3-yl}benzonitrile

A solution of the compound described in Example 30 (1 mmol), (3-cyano)phenylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 63

### 3-(2-chlorophenyl)-5-[(2-methylpyrrolidin-1-yl)carbonyl]pyridine

A solution of the compound described in Example 30 (1 mmol), 2-chlorophenylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 64

### 3-(3,4-dimethylphenyl)-5-[(2-methylpyrrolidin-1-yl)carbonyl]pyridine

A solution of the compound described in Example 30, 3,4-dimethylphenylboronic acid (2.0 mmol), and tetrakis(triphenylphosphine)palladium (0.05 mmol) in dichloromethane (1.5 mL) and ethanol (0.25 mL) was treated with 2 M sodium carbonate (0.5 mL), heated to 87° C. overnight, and concentrated. The residue was dissolved in diethyl ether, washed with water three times, dried (Na₂SO₄), filtered, and concentrated. The concentrate was purified by HPLC using a C-18 column with a solvent system increasing in gradient over 50 minutes from 5% to 100% acetonitrile/water containing 0.01% TFA and lyophilized to provide the desired product as the trifluoroacetate salt. MS m/e 295 (M+H)⁺; ¹H NMR (DMSO-d₆) δ 0.87 (d, 0.75H), 1.28 (d, 2.25H), 1.51-1.63 (m, 1H), 1.69-1.80 (m, 1H), 1.83-2.00 (m, 1H), 2.03-2.15 (m, 1H), 2.29 (d, 6H), 3.33-3.44 (m, 0.75H), 3.50-3.63 (m, 1.25H), 3.99 (br s, 0.25H), 4.15-4.24 (m, 0.75H), 7.27 (d, 1H), 7.50 (dd, 1H), 7.57 (s, 1H), 8.10 (t, 1H), 8.57-8.65 (m, 1H), 8.92 (d, 1H).

### EXAMPLE 65

### 3-(3-ethoxyphenyl)-5-[(2-methylpyrrolidin-1-yl)carbonyl]pyridine

A solution of the compound described in Example 30 (1 mmol), 3-ethoxyphenylboronic acid (2.0 mmol), and tetrakis(triphenylphosphine)palladium (0) (0.05 mmol) in dichloromethane (1.5 mL) and ethanol (0.25 mL) was treated with 2 M sodium carbonate (0.5 mL), heated to 87° C. overnight, and concentrated. The residue was dissolved in diethyl ether, washed three times with water, dried (Na₂SO₄), filtered, and concentrated. The concentrate was purified by HPLC using a C-18 column with a solvent system increasing in gradient over 50 minutes from 5% to 100% acetonitrile/water containing 0.01% TFA and lyophilized to provide the desired product as the trifluoroacetate salt. MS m/e 268 (M+H)⁺; ¹H NMR (DMSO-d₆) δ 0.87 (d, 0.75H), 1.27 (d, 2.25H), 1.53-1.64 (m, 1H), 1.67-1.80 (m, 1H), 1.82-1.99 (m, 1H), 2.04-2.15 (m, 1H), 3.32-3.40 (m, 0.75H), 3.49-3.61 (m, 1.25H), 4.01 (br s, 0.25H), 4.14-4.26 (m, 0.75H), 7.85 (d, 2H), 8.28-8.34 (m, 1H), 8.70 (dd, 2H), 8.72-8.78 (m, 1H), 9.09 (d, 1H).

### EXAMPLE 66

### 5-[(2-methylpyrrolidin-1-ylcarbonyl]-3,4′-bipyridine

A solution of the compound described in Example 30 (1 mmol), 4-pyridylboronic acid (2.0 mmol), and tetrakis(triphenylphosphine)palladium (0) (0.05 mmol) in dichloromethane (1.5 mL) and ethanol (0.25 mL) was treated with 2 M sodium carbonate (0.5 mL), heated to 87° C. overnight, and concentrated. The concentrate was dissolved in diethyl ether, washed three times with water, dried (Na₂SO₄), filtered, and concentrated. The concentrate was purified by HPLC using a C-18 column with a solvent system increasing in gradient over 50 minutes from 5% to 100% acetonitrile/water containing 0.01% TFA and lyophilized to provide the desired product as the trifluoroacetate salt. MS m/e 268 (M+H)⁺; ¹H NMR (DMSO-d₆) δ 0.87 (d, 0.75H), 1.27 (d, 2.25H), 1.53-1.64 (m, 1H), 1.67-1.80 (m, 1H), 1.82-1.99 (m, 1H), 2.04-2.15 (m, 1H), 3.32-3.40 (m, 0.75H), 3.49-3.61 (m, 1.25H), 4.01 (br s, 0.25H), 4.14-4.26 (m, 0.75H), 7.85 (d, 2H), 8.28-8.34 (m, 1H), 8.70 (dd, 2H), 8.72-8.78 (m, 1H), 9.09 (d, 1H).

### EXAMPLE 67

### 3-(3-furyl)-5-[(2-methylpyrrolidin-1-yl)carbonyl]pyridine

A solution of the compound described in Example 30 (1 mmol), 3-furylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 68

### 2-(cyclohexylmethyl)-5-[(2-methylpyrrolidin-1-yl)carbonyl]pyridine

A solution of the compound described in Example 31 (1 mmol), cyclohexylmethylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 69

### 7-{5-[(2-methylpyrrolidin-1-yl)carbonyl]pyridin-2-yl}heptanenitrile

A solution of the compound described in Example 31 (1 mmol), 6-cyanohexylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 70

### 2-hexyl-5-[(2-methylpyrrolidin-1-yl)carbonyl]pyridine

A solution of the compound described in Example 31 (1 mmol), hexylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 71

### 2-bicyclo[2.2.1]hept-2-yl-5-[(2-methylpyrrolidin-1-yl)carbonyl]pyridine

A solution of the compound described in Example 31 (1 mmol), 2-norbornylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 72

### 2-(1-methylpentyl)-5-[(2-methylpyrrolidin-1-yl)carbonyl]pyridine

A solution of the compound described in Example 31 (1 mmol), 1-methylpen-1-tylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 73

### 5-[(2-methylpyrrolidin-1-yl)carbonyl]-2-thien-2-ylpyridine

A solution of the compound described in Example 31 (1 mmol), 2-thienylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 74

### 2-(3,5-dichlorophenyl)-5-[(2-methylpyrrolidin-1-yl)carbonyl]pyridine

A solution of the compound described in Example 31 (1 mmol), 3,5-dichlorophenylboronic acid (2.0 mmol), and tetrakis(triphenylphosphine)palladium (0) (0.05 mmol) in dichloromethane (1.5 mL) and ethanol (0.25 mL) is treated with 2 M sodium carbonate (0.5 mL), heated to 87° C. overnight, and concentrated. The concentrate is dissolved in diethyl ether, washed three times with water, dried (Na₂SO₄), filtered, and concentrated. The concentrate is purified by HPLC using a C-18 column with a solvent system increasing in gradient over 50 minutes from 5% to 100% acetonitrile/water containing 0.01% TFA and lyophilized to provide the desired product as the trifluoroacetate salt.

### EXAMPLE 75

### 1-[(2-chloro-6-methyl-3-pyridinyl)carbonyl]-3-piperidinecarboxamide

The desired product was prepared by substituting 2-chloro-6-methylnicotinic acid for 6-methylnicotinic acid and nipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether and treated dropwise with 1.0 M HCl in diethyl ether. The precipitate was isolated by filtration to provide the desired product as the hydrochloride salt. MS m/e 282 (M+H)⁺; ¹H NMR (DMSO-d₆) δ 1.24-1.70 (m, 2.5H), 1.73-1.81 (m, 0.5H), 1.85-2.02 (m, 1H), 2.16-2.39 (m, 1H), 2.48 (s, 3H), 2.60-2.73 (m, 0.25H), 2.76-2.88 (m, 0.5H), 2.91-3.26 (br m, 2.25H), 4.20 (br d, 0.2H), 4.48 (br d, 0.8H), 6.78-6.93 (br m, 1H), 7.26 (br d, 0.5H), 7.32-7.47 (m, 1.5H), 7.68-7.79 (m, 1H).

### EXAMPLE 76

### 1-[(2-chloro-6-methyl-3-pyridinyl)carbonyl]-N,N-diethyl-3-piperidinecarboxamide

The desired product was prepared by substituting 2-chloro-6-methylnicotinic acid for 6-methylnicotinic acid and N,N-diethylnipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether and treated dropwise with 1.0 M HCl in diethyl ether. The precipitate was isolated by filtration to provide the desired product as the hydrochloride salt. MS m/e 338 (M+H)⁺; ¹H NMR (DMSO-d₆) δ 0.88-0.99 (m, 3H), 1.02 (t, 1.5H), 1.16 (t, 1.5H), 1.36-1.88 (m, 4H), 2.48 (d, 3H), 2.60-2.95 (m, 2H), 2.96-3.18 (m, 3H), 3.19-3.45 (m, 3H), 4.35-4.56 (br m, 1H), 7.33-7.40 (m, 1H), 7.71 (d, 0.5H), 7.82-7.91 (m, 0.5H).

### EXAMPLE 77

### 2-methyl-5-(1-pyrrolidinylcarbonyl)pyridine

The desired product was prepared by substituting pyrrolidine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether and treated dropwise with 1.0 M HCl in diethyl ether. The precipitate was isolated by filtration to provide the desired product as the hydrochloride salt. MS m/e 191.1 (M+H)⁺; ¹H NMR (DMSO-d₆) δ 1.78-1.95 (m, 4H), 2.70 (s, 3H), 3.39-3.53 (m, 4H), 7.78 (d, 1H), 8.37 (dd, 1H), 8.85 (d, 1H).

### EXAMPLE 78

### 1-(3-pyridinylcarbonyl)-3-piperidinecarboxamide

The desired product was prepared by substituting nicotinic acid for 6-methylnicotinic acid and nipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 233 (M+H)⁺; ¹H NMR (DMSO-d₆) δ 1.44 (br s, 1H), 1.53-1.81 (br m, 2H), 1.85-2.00 (br m, 1H), 2.25-2.40 (br m, 1H), 2.75-3.26 (br m, 2H), 3.47 (br s, 1H), 4.24 (br s, 0.5H), 4.45 (br s, 0.5H), 6.84 (br d, 1H), 7.32 (br d, 1H), 7.51 (dd, 1H), 7.86 (d, 1H), 8.61 (s, 1H), 8.68 (dd, 1H).

### EXAMPLE 79

### 1-(4-fluorophenyl)-4-(3-pyridinylcarbonyl)piperazine

The desired product was prepared by substituting nicotinic acid for 6-methylnicotinic acid and 1-(4-fluorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 286 (M+H)⁺; ¹H NMR (DMSO-d₆) δ 3.13 (br d, 4H), 3.48 (br s, 2H), 3.77 (br s, 2H), 6.94-7.02 (m, 2H), 7.03-7.11 (m, 2H), 7.51 (dd, 1H), 7.87-7.91 (m, 1H), 8.59-8.73 (m, 2H).

### EXAMPLE 80

### 3-[(2-methyl-1-pyrrolidinyl)carbonyl]pyridine

The desired product was prepared by substituting nicotinic acid for 6-methylnicotinic acid in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 191.1 (M+H)⁺; ¹H NMR (DMSO-d₆) δ 0.86 (d, 0.6H), 1.27 (d, 2.4H), 1.50-1.65 (m, 1H), 1.66-1.82 (m, 1H), 1.83-2.16 (m, 2H), 3.29-3.41 (m, 0.75H), 3.45-3.60 (m, 1.25H), 3.89-4.02 (m, 0.25H), 4.10-4.24 (m, 0.75H), 7.91 (dd, 1H), 8.37-8.50 (m, 1H), 8.87 (d, 1H), 8.97 (d, 1H).

### EXAMPLE 82

### 3-(2-bromophenyl)-5-[(2-methyl-1-pyrrolidinyl)carbonyl]pyridine

The desired product was prepared by substituting 2-bromophenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 346.1 (M+H)⁺; ¹H NMR (DMSO-d₆) δ 0.89 (d, 0.75H), 1.26 (d, 2.25H), 1.51-1.63 (m, 1H), 1.69-1.81 (m, 1H), 1.84-1.97 (m, 1H), 2.04-2.14 (m, 1H), 3.34-3.43 (m, 0.6H), 3.50-3.61 (m, 1.4H), 4.00-4.09 (m, 0.25H), 4.13-4.23 (m, 0.75H), 7.37-7.44 (m, 1H), 7.47-7.57 (m, 2.5H), 7.59-7.65 (m, 0.5H), 7.80 (d, 1H), 7.94 (s, 1H), 8.64-8.74 (m, 1H).

### EXAMPLE 83

### 3-(2-methylphenyl)-5-[(2-methyl-1-pyrrolidinyl)carbonyl]pyridine

The desired product was prepared by substituting 2-methylphenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 281.1 (M+H)⁺; ¹H NMR (DMSO-d₆) δ 0.88 (d, 0.8H), 1.29 (d, 2.2H), 1.51-1.64 (m, 1H), 1.69-1.79 (m, 1H), 1.84-1.95 (m, 1H), 2.04-2.13 (m, 1H), 3.34-3.42 (m, 0.7H), 3.50-3.59 (m, 1.3H), 3.96-4.04 (m, 0.25H), 4.14-4.23 (m, 0.75H), 7.25-7.38 (m, 4H), 7.87 (t, 1H), 8.59-8.70 (m, 2H).

### EXAMPLE 84

### 3-(4-methylphenyl)-5-[(2-methyl-1-pyrrolidinyl)carbonyl]pyridine

The desired product was prepared by substituting 4-methylphenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 281.1 (M+H)⁺; ¹H NMR (DMSO-d₆) δ 0.88 (d, 0.75H), 1.29 (d, 2.25H), 1.52-1.62 (m, 1H), 1.68-1.79 (m, 1H), 1.84-1.94 (m, 1H), 2.05-2.13 (m, 1H), 3.32-3.41 (m, 0.7H), 3.50-3.62 (m, 1.3H), 3.96-4.04 (m, 0.25H), 4.14-4.24 (m, 0.75H), 7.33 (d, 3H), 7.68 (d, 2H), 8.11 (t, 1H), 8.58-8.66 (m, 1H), 8.93 (d, 1H).

### EXAMPLE 85

### 4-{5-[(2-methyl-1-pyrrolidinyl)carbonyl]-3-pyridinyl}benzoic acid

The desired product was prepared by substituting 4-(carbomethoxy)phenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 311.1 (M+H)⁺; ¹H NMR (DMSO-d₆) δ 0.88 (d, 0.75H), 1.29 (d, 2.25H), 1.54-1.62 (m, 1H), 1.69-1.80 (m, 1H), 1.85-1.99 (m, 1H), 2.05-2.14 (m, 1H), 3.33-3.42 (m, 0.75H), 3.51-3.61 (m, 1.25H), 3.98-4.06 (m, 0.25H), 4.15-4.24 (m, 0.75H), 7.79 (d, 2H), 8.00 (d, 2H), 8.16-8.60 (m, 1H), 8.62-8.69 (m, 1H), 8.98 (d, 1H).

### EXAMPLE 86

### 4-{5-[(2-methyl-1-pyrrolidinyl)carbonyl]-3-pyridinyl}aniline

The desired product was prepared by substituting 4-(amino)phenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 282.1 (M+H)⁺; ¹H NMR (DMSO-d₆) δ 0.87 (d, 0.75H), 1.20-1.30 (m, 2.25H), 1.51-1.60 (m, 1H), 1.68-1.79 (m, 1H), 1.81-1.95 (m, 1H), 2.03-2.13 (m, 1H), 3.31-3.40 (m, 0.75H), 3.47-3.60 (m, 1.25H), 3.93-4.04 (m, 0.25H), 4.12-4.23 (m, 0.75H), 5.36 (s, 2H), 6.67 (d, 2H), 7.47 (d, 2H), 7.96 (t, 1H), 8.43-8.50 (m, 1H), 8.83 (d, 1H).

### EXAMPLE 87

### 3-{5-[(2-methyl-1-pyrrolidinyl)carbonyl]-3-pyridinyl}phenol

The desired product was prepared by substituting 3-(hydroxy)phenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 283 (M+H)⁺; ¹H NMR (DMSO-d₆) δ 0.88 (d, 0.75H), 1.28 (d, 2.25H), 1.52-1.61 (m, 1H), 1.69-1.81 (m, 1H), 1.85-1.98 (m, 1H), 2.04-2.15 (m, 1H), 3.33-3.43 (m, 0.75H), 3.51-3.60 (m, 1.25H), 3.96-4.04 (m, 0.25H), 4.15-4.24 (m, 0.75H), 6.85 (dd, 1H), 7.1 (t, 1H), 7.17 (d, 1H), 7.31 (t, 1H), 8.06 (t, 1H), 8.59-8.67 (br m, 1H), 8.88 (d, 1H).

### EXAMPLE 88

### 3-{5-[(2-methyl-1-pyrrolidinyl)carbonyl]-3-pyridinyl}benzonitrile

The desired product was prepared by substituting 3-(cyano)phenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 292 (M+H)⁺; ¹H NMR (DMSO-d₆) δ 0.87 (d, 0.75H), 1.29 (d, 2.25H), 1.53-1.63 (m, 1H), 1.69-1.81 (m, 1H), 1.85-1.98 (m, 1H), 2.04-2.15 (m, 1H), 3.33-3.40 (m, 0.75H), 3.50-3.61 (m, 1.25H), 3.97-4.07 (m, 0.25H), 4.15-4.25 (m, 0.75H), 7.72 (t, 1H), 7.88-7.93 (m, 1H), 8.14-8.19 (m, 1H), 8.25-8.30 (br m, 1H), 8.33 (t, 1H), 8.66-8.73 (br m, 1H), 9.04 (d, 1H).

### EXAMPLE 89

### 3-[(2-methyl-1-pyrrolidinyl)carbonyl]-5-[3-(trifluoromethyl)phenyl]pyridine

The desired product was prepared by substituting 3-(trifluoromethyl)phenylboronic acid for phenylboronic acid in Example 58. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 335 (M+H)⁺; ¹H NMR (DMSO-d₆) δ 0.88 (d, 0.75H), 1.29 (d, 2.25H), 1.52-1.61 (m, 1H), 1.68-1.80 (m, 1H), 1.83-1.96 (m, 1H), 2.02-2.11 (m, 1H), 3.33-3.44 (m, 0.75H), 3.50-3.62 (m, 1.25H), 3.99-4.06 (m, 0.25H), 4.13-4.21 (m, 0.75H), 7.73-7.84 (m, 2H), 8.09-8.17 (m, 2H), 8.25-8.32 (m, 1H), 8.67-8.73 (m, 1H), 9.02-9.07 (m, 1H).

### EXAMPLE 90

### 1-(4-fluorophenyl)-4-{[6-(1H-pyrazol-1-yl)-3-pyridinyl]carbonyl}piperazine

The desired product was prepared by substituting 6-pyrazolylnicotinic acid for 6-methylnicotinic acid and 1-(4-fluorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 352 (M+H)⁺; ¹H NMR (DMSO-d₆) δ 3.15 (br s, 4H), 3.68 (br d, 4H), 6.22 (dd, 1H), 6.96-7.02 (m, 2H), 7.04-7.10 (m, 2H), 7.87-7.89 (m, 1H), 7.99 (dd, 1H), 8.08 (dd, 1H), 8.57 (dd, 1H), 8.66 (dd, 1H).

### EXAMPLE 91

### N-methyl-5-[(2-methyl-1-pyrrolidinyl)carbonyl]-N-(tetrahydro-2-furanylmethyl)-2-pyridinamine

A solution of 2-chloro-5-[(2-methyl-1-pyrrolidinyl)carbonyl]pyridine (1.0 mmol), N-methyl-N-(tetrahydro-2-furanylmethyl)amine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 304 (M+H)⁺; ¹H NMR (CDCl₃) δ 1.33 (br s, 3H), 1.58-2.13 (m, 7H), 2.14-2.23 (m, 1H), 3.26 (s, 3H), 3.51-3.84 (m, 5H), 4.18-4.29 (m, 2H), 7.07 (d, 1H), 7.93 (d, 1H), 8.19 (d, 1H).

### EXAMPLE 92

### N,N-diethyl-N′-methyl-N′-{5-[(2-methyl-1-pyrrolidinyl)carbonyl]-2-pyridinyl}-1,2-ethanediamine

A solution of 2-chloro-5-[(2-methyl-1-pyrrolidinyl)carbonyl]pyridine (1.0 mmol), N,N-diethyl-N′-methyl-1,2-ethanediamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 319 (M+H)⁺; ¹H NMR (CDCl₃) δ 1.30-1.40 (m, 9H), 1.68 (br s, 1H), 1.82 (br s, 1H), 2.00 (br s, 1H), 2.14-2.23 (m, 1H), 3.26 (s, 3H), 3.32-3.39 (m, 4H), 3.45 (t, 2H), 3.54 (br s, 1H), 4.08 (t, 2H), 4.19-4.30 (br m, 1H), 7.12 (d, 1H), 8.01 (d, 1H), 8.28 (d, 1H).

### EXAMPLE 93

### N-methyl-5-[(2-methyl-1-pyrrolidinyl)carbonyl]-N-[2-(2-pyridinyl)ethyl]-2-pyridinamine

A solution of 2-chloro-5-[(2-methyl-1-pyrrolidinyl)carbonyl]pyridine (1.0 mmol), N-methyl-N-[2-(2-pyridinyl)ethyl]amine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 325 (M+H)⁺; ¹H NMR (CDCl₃) δ 1.33 (br s, 3H), 1.68 (br s, 1H), 1.83 (br s, 1H), 2.01 (br s, 1H), 2.14-2.24 (m, 1H), 3.28 (s, 3H), 3.46 (t, 2H), 3.54 (br s, 1H), 3.60-3.69 (m, 1H), 4.17 (t, 2H), 4.25 (br s, 1H), 7.18 (d, 1H), 7.89-7.94 (m, 1H), 7.99-8.08 (m, 2H), 8.16 (d, 1H), 8.46-8.51 (m, 1H), 8.76 (dd, 1H).

### EXAMPLE 94

### 1-methyl-4-{5-[(2-methyl-1-pyrrolidinyl)carbonyl]-2-pyridinyl}piperazine

A solution of 2-chloro-5-[(2-methyl-1-pyrrolidinyl)carbonyl]pyridine (1.0 mmol), 1-methylpiperazine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 289 (M+H)⁺; ¹H NMR (CDCl₃) δ 1.23-1.42 (br m, 3H), 1.62-1.73 (br m, 1H), 1.75-1.87 (br m, 1H), 1.94-2.06 (br m, 1H), 2.14-2.23 (m, 1H), 2.98 (s, 3H), 3.42 (br s, 1.5H), 3.35-3.75 (br m, 6.5H), 4.26 (br s, 1H), 4.57 (br s, 2H), 7.15 (d, 1H), 7.96 (d, 1H), 8.35 (s, 1H).

### EXAMPLE 95

### 1-ethyl-4-{5-[(2-methyl-1-pyrrolidinyl)carbonyl]-2-pyridinyl}piperazine

A solution of 2-chloro-5-[(2-methyl-1-pyrrolidinyl)carbonyl]pyridine (1.0 mmol), 1-ethylpiperazine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 303 (M+H)⁺; ¹H NMR (CDCl₃) δ 1.27-1.38 (br m, 3H), 1.41 (t, 3H), 1.62-1.73 (br m, 1H), 1.75-1.88 (br m, 1H), 1.93-2.08 (br m, 1H), 2.14-2.24 (m, 1H), 3.14-3.25 (br m, 1.5H), 3.26-3.34 (m, 2H), 3.39-3.78 (br m, 6.5H), 4.26 (br s, 1H), 4.57 (br d, 2H), 7.18 (d, 1H), 7.99 (d, 1H), 8.34 (s, 1H).

### EXAMPLE 96

### 1-{5-[(2-methyl-1-pyrrolidinyl)carbonyl]-2-pyridinyl}-4-(2-pyridinyl)piperazine

A solution of 2-chloro-5-[(2-methyl-1-pyrrolidinyl)carbonyl]pyridine (1.0 mmol), 1-(pyridin-2-yl)piperazine (5.0 mmol) and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 352 (M+H)⁺; ¹H NMR (CDCl₃) δ 1.34 (br s, 3H), 1.68 (br s, 1H), 1.82 (br s, 1H), 1.95-2.07 (br m, 1H), 2.15-2.23 (m, 1H), 3.55 (br s, 1H), 3.62-3.69 (m, 1H), 3.99-4.08 (m, 8H), 4.26 (br s, 1H), 7.04-7.09 (m, 1H), 7.15 (d, 1H), 7.42 (d, 1H), 7.98-8.04 (m, 2H), 8.08-8.12 (m, 1H), 8.30 (d, 1H).

### EXAMPLE 97

### 1-benzyl-4-{5-[(2-methyl-1-pyrrolidinyl)carbonyl]-2-pyridinyl}piperazine

A solution of 2-chloro-5-[(2-methyl-1-pyrrolidinyl)carbonyl]pyridine (1.0 mmol), 1-benzylpiperazine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt MS m/e 365 (M+H)⁺; ¹H NMR (CDCl₃) δ 1.33 (br s, 3H), 1.67 (br s, 1H), 1.80 (br s, 1H), 1.94-2.07 (br m, 1H), 2.12-2.22 (m, 1H), 3.27 (br s, 1.5H), 3.33-3.67 (br m, 6.5H), 4.25 (br s, 1H), 4.43 (s, 2H), 4.57 (br s, 2H), 7.08 (d, 1H), 7.50-7.61 (m, 5H), 7.91 (br d, 1H), 8.35 (s, 1H).

### EXAMPLE 98

### 1-(2-methoxyphenyl)-4-{5-[(2-methyl-1-pyrrolidinyl)carbonyl]-2-pyridinyl}piperazine

A solution of 2-chloro-5-[(2-methyl-1-pyrrolidinyl)carbonyl]pyridine (1.0 mmol), 1-(2-methoxyphenyl)piperazine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 381 (M+H)⁺; ¹H NMR (CDCl₃) δ 1.24-1.42 (br m, 3H), 1.68 (br s, 1H), 1.82 (br s, 1H), 1.95-2.08 (br m, 1H), 2.15-2.24 (m, 1H), 3.50-3.71 (br m, 6H), 3.94-4.15 (br m, 7H), 4.26 (br s, 1H), 7.09 (t, 1H), 7.22 (dd, 2H), 7.34-7.47 (m, 2H), 7.99 (br d, 1H), 8.30 (d, 1H).

### EXAMPLE 99

### 1-methyl-4-{5-[(2-methyl-1-pyrrolidinyl)carbonyl]-2-pyridinyl}-1,4-diazepane

A solution of 2-chloro-5-[(2-methyl-1-pyrrolidinyl)carbonyl]pyridine (1.0 mmol), 1-methyl-1,4-diazepane (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 303 (M+H)⁺; ¹H NMR (CDCl₃) δ 1.27-1.41 (br m, 3H), 1.68 (br s, 1H), 1.82 (br s, 1H), 1.95-2.06 (br m, 1H), 2.14-2.24 (m, 1H), 2.34-2.45 (br m, 2H), 3.34-3.46 (br m, 2H), 3.49-3.70 (br m, 3H), 3.72-3.90 (br m, 3H), 3.97-4.07 (br m, 1H), 4.19-4.35 (br m, 2H), 7.24 (d, 1H), 8.09 (brd, 1H), 8.27 (d, 1H).

### EXAMPLE 100

### N-ethyl-N-methyl-5-[(2-methyl-1-pyrrolidinyl)carbonyl]-2-pyridinamine

A solution of 2-chloro-5-[(2-methyl-1-pyrrolidinyl)carbonyl]pyridine (1.0 mmol), N-ethyl-N-methylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 248 (M+H)⁺; ¹H NMR (CDCl₃) δ 1.18-1.43 (m, 6H), 1.68 (br s, 1H), 1.83 (br s, 1H), 2.01 (br s, 1H), 2.14-2.24 (m, 1H), 3.30 (s, 3H), 3.50-3.61 (br m, 1H), 3.62-3.69 (m 1H), 3.73 (q, 2H), 4.19-4.30 (br m, 1H), 7.29 (d, 1H), 8.07-8.15 (m, 2H).

### EXAMPLE 101

### N-butyl-N-methyl-5-[(2-methyl-1-pyrrolidinylcarbonyl]-2-pyridinamine

A solution of 2-chloro-5-[(2-methyl-1-pyrrolidinyl)carbonyl]pyridine (1.0 mmol), N-butyl-N-methylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 276 (M+H)⁺; ¹H NMR (CDCl₃) δ 1.01 (t, 3H), 1.26-1.38 (br m, 3H), 1.38-1.49 (m, 2H), 1.63-1.74 (m, 3H), 1.83 (br s, 1H), 2.01 (br s, 1H), 2.15-2.24 (m, 1H), 3.30 (s, 3H), 3.52-3.60 (br m, 1H), 3.61-3.69 (m, 3H), 4.19-4.30 (br m, 1H), 7.27 (d, 1H), 8.06-8.13 (m, 2H).

### EXAMPLE 102

### N-isobutyl-N-methyl-5-[(2-methyl-1-pyrrolidinyl)carbonyl]-2-pyridinamine

A solution of 2-chloro-5-[(2-methyl-1-pyrrolidinyl)carbonyl]pyridine (1.0 mmol), N-isobutyl-N-methylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 276 (M+H)⁺; ¹H NMR (CDCl₃) δ 1.01 (d, 6H), 1.34 (br d, 3H), 1.68 (br s, 1H), 1.83 (br s, 1H), 2.02 (br s, 1H), 2.10-2.24 (m, 1H), 3.31 (s, 3H), 3.52 (d, 2H), 3.57 (br s, 1H), 3.62-3.70 (m, 1H), 4.25 (br s, 1H), 7.31 (d, 1H), 8.07-8.13 (m, 2H).

### EXAMPLE 103

### N-methyl-5-[(2-methyl-1-pyrrolidinyl)carbonyl]-N-pentyl-2-pyridinamine

A solution of 2-chloro-5-[(2-methyl-1-pyrrolidinyl)carbonyl]pyridine (1.0 mmol), N-pentyl-N-methylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 290 (M+H)⁺; ¹H NMR (CDCl₃) δ 0.95 (t, 3H), 1.26-1.48 (m, 7H), 1.63-1.76 (m, 3H). 1.83 (br s, 1H), 2.01 (br s, 1H), 2.15-2.24 (m, 1H), 3.29 (s, 3H), 3.56 (br s, 1H), 3.61-3.70 (m, 3H), 4.25 (br s, 1H), 7.27 (d, 1H), 8.05-8.13 (m, 2H).

### EXAMPLE 104

### N-cyclohexyl-N-methyl-5-[(2-methyl-1-pyrrolidinyl)carbonyl]-2-pyridinamine

A solution of 2-chloro-5-[(2-methyl-1-pyrrolidinyl)carbonyl]pyridine (1.0 mmol), N-cyclohexyl-N-methylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 302 (M+H)⁺; ¹H NMR (CDCl₃) δ 1.21-1.39 (m, 4H), 1.47-1.59 (m, 2H), 1.65-1.78 (m, 4H), 1.81-1.96 (m, 5H), 2.01 (br s, 1H), 2.14-2.24 (m, 1H), 3.15 (s, 3H), 3.56 (br s, 1H), 3.62-3.70 (m, 1H), 3.98-4.07 (m, 1H), 4.20-4.30 (br m, 1H), 7.27 (d, 1H), 8.07-8.14 (m, 2H).

### EXAMPLE 105

### 5-[(2-methyl-1-pyrrolidinyl)carbonyl]-N,N-dipropyl-2-pyridinamine

A solution of 2-chloro-5-[(2-methyl-1-pyrrolidinyl)carbonyl]pyridine (1.0 mmol), N,N-dipropylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 290 (M+H)⁺; ¹H NMR (CDCl₃) δ 1.03 (t, 6H), 1.25-1.40 (br m, 3H), 1.62-1.78 (m, 5H), 1.83 (br s, 1H), 2.01 (br s, 1H), 2.15-2.24 (m, 1H), 3.59 (t, 5H), 3.62-3.69 (m, 1H), 4.19-4.29 (br m, 1H), 7.24 (d, 1H), 8.04-8.11 (m, 2H).

### EXAMPLE 106

### N,N-dibutyl-5-[(2-methyl-1-pyrrolidinyl)carbonyl]-2-pyridinamine

A solution of 2-chloro-5-[(2-methyl-1-pyrrolidinyl)carbonyl]pyridine (1.0 mmol), N,N-dibutylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 318 (M+H)⁺; ¹H NMR (CDCl₃) δ 1.01 (t, 6H), 1.33 (br d, 3H), 1.39-1.49 (m, 4H), 1.62-1.73 (m, 5H), 1.83 (br s, 1H), 2.01 (br s, 1H), 2.14-2.24 (m, 1H), 3.52-3.70 (m, 6H), 4.20-4.30 (br m, 1H), 7.22 (d, 1H), 8.05-8.12 (m, 2H).

### EXAMPLE 107

### 5-[(2-methyl-1-pyrrolidinyl)carbonyl]-2-(1-pyrrolidinyl)pyridine

A solution of 2-chloro-5-[(2-methyl-1-pyrrolidinyl)carbonyl]pyridine (1.0 mmol), pyrrolidine (5.0 mmol) and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 260 (M+H)⁺; ¹H NMR (CDCl₃) δ 1.34 (br d, 3H), 1.68 (br s, 1H), 1.89 (br s, 1H), 1.96-2.07 (br m, 1H), 3.13-2.24 (m, 5H), 3.55 (br s, 1H), 3.60-3.71 (m, 5H), 4.20-4.30 (br m, 1H), 7.13 (d, 1H), 8.05-8.13 (m, 2H).

### EXAMPLE 108

### 2-(2-methyl-1-pyrrolidinyl)-5-[(2-methyl-1-pyrrolidinyl)carbonyl]pyridine

A solution of 2-chloro-5-[(2-methyl-1-pyrrolidinyl)carbonyl]pyridine (1.0 mmol), 2-methylpyrrolidine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 274 (M+H)⁺; ¹H NMR (CDCl₃) δ 1.26-1.40 (m, 6H), 1.68 (br s, 1H), 1.94 (br s, 1H), 1.91-2.08 (br m, 2H), 2.15-2.35 (m, 4H), 3.51-3.60 (m, 2H), 3.61-3.69 (m, 1H), 3.77 (tr, 1H), 4.19-4.29 (br m, 1H), 4.30-4.38 (m, 1H), 7.18 (br d, 1H), 8.06-8.13 (m, 2H).

### EXAMPLE 109

### 5-[(2-methyl-1-pyrrolidinyl)carbonyl]-2-(1-piperidinyl)pyridine

A solution of 2-chloro-5-[(2-methyl-1-pyrrolidinyl)carbonyl]pyridine (1.0 mmol), piperidine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 274 (M+H)⁺; ¹H NMR (CDCl₃) δ 1.33 (br d, 3H), 1.67 (br s, 1H), 1.75-1.88 (m, 7H), 1.96-2.06 (br m, 1H), 2.15-2.23 (m, 1H), 3.56 (br s, 1H), 3.61-3.69 (m, 1H), 3.72-3.79 (m, 4H), 4.20-4.30 (br m, 1H), 7.39 (d, 1H), 8.06-8.14 (m, 2H).

### EXAMPLE 110

### 2-(4-methyl-1-piperidinyl)-5-[(2-methyl-1-pyrrolidinyl)carbonyl]pyridine

A solution of 2-chloro-5-[(2-methyl-1-pyrrolidinyl)carbonyl]pyridine (1.0 mmol), 4-methylpiperidine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 288 (M+H)⁺; ¹H NMR (CDCl₃) δ 1.03 (d, 3H), 1.28-1.38 (m, 5H), 1.67 (br s, 1H), 1.78-1.94 (br m, 4H), 2.02 (br s, 1H), 2.14-2.24 (m, 1H), 3.25-3.34 (m, 2H), 3.56 (br s, 1H), 3.61-3.69 (m, 1H), 4.18-4.29 (m, 3H), 7.39 (d, 1H), 8.05-8.13 (m, 2H).

### EXAMPLE 111

### N-(2-methoxyethyl)-5[(2-methyl-1-pyrrolidinyl)carbonyl]-N-propyl-2-pyridinamine

A solution of 2-chloro-5-[(2-methyl-1-pyrrolidinyl)carbonyl]pyridine (1.0 mmol), N-(2-methoxyethyl)-N-propylamine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 306 (M+H)⁺; ¹H NMR (CDCl₃) δ 1.02 (t, 3H), 1.34 (br d, 3H), 1.62-1.78 (m, 3H), 1.83 (br s, 1H), 2.01 (br s, 1H), 2.14-2.24 (m, 1H), 3.36 (s, 3H), 3.57 (br s, 1H), 3.59-3.67 (m, 3H), 3.70 (t, 2H), 3.86 (t, 2H), 4.20-4.30 (br m, 1H), 7.30 (d, 1H), 8.03-8.15 (m, 2H).

### EXAMPLE 112

### N,N-bis(2-methoxyethyl)-5-[(2-methyl-1-pyrrolidinyl)carbonyl]-2-pyridinamine

A solution of 2-chloro-5-[(2-methyl-1-pyrrolidinyl)carbonyl]pyridine (1.0 mmol), N,N-bis(2-methoxyethyl)amine (5.0 mmol), and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 322 (M+H)⁺; ¹H NMR (CDCl₃) δ 1.33 (br d, 3H), 1.62-1.73 (br m, 1H), 1.83 (br s, 1H), 2.01 (br s, 1H), 2.14-2.24 (m, 1H), 3.36 (s, 6H), 3.56 (br s, 1H), 3.61-3.75 (m, 5H), 3.91 (t, 4H), 4.19-4.29 (br m, 1H), 7.37 (d, 1H), 8.08 (d, 1H), 8.13 (d, 1H).

### EXAMPLE 113

### 4-{5-[(2-methyl-1-pyrrolidinyl)carbonyl]-2-pyridinyl}morpholine

A solution of 2-chloro-5-[(2-methyl-1-pyrrolidinyl)carbonyl]pyridine (1.0 mmol), morpholine (5.0 mmol) and triethylamine (5.0 mmol) in N-methylpyrrolidinone (5 mL) was heated to 150° C. for 24 hours and concentrated in vacuo. The residue was purified by HPLC using a C-18 column and a solvent system varying in a gradient from 10% to 50% acetonitrile/water containing 0.1% TFA over 50 minutes then lyophilized to provide the desired product as the trifluoroacetate salt. This was dissolved in dichloromethane and shaken with basic resin MP carbonate for four hours. The resin was removed by filtration and the filtrate was concentrated in vacuo. The concentrate was dissolved in diethyl ether/methanol and treated dropwise with 1.0 M HCl in diethyl ether. The precipitate was isolated by filtration to provide the desired product as the hydrochloride salt. MS m/e 276 (M+H)⁺; ¹H NMR (CDCl₃) δ 1.34 (br d, 3H), 1.63-1.73 (br m, 1H). 1.78-1.90 (br m, 1H), 1.96-2.08 (br m, 1H), 2.15-2.24 (m, 1H), 3.55 (br s, 1H), 3.61-3.69 (m, 1H), 3.72 (t, 4H), 3.87 (t, 4H), 4.20-4.30 (br m, 1H), 7.38 (d, 1H), 8.11-8.19 (m, 2H).

### EXAMPLE 114

### (3R)-1-{[2-methyl-6-(trifluoromethyl)-3-pyridinyl]carbonyl}-3-piperidinol

The desired product was prepared by substituting 2-methyl-6-(trifluoromethyl)nicotinic acid for 6-methylnicotinic acid and (3R)-3-piperidinol for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product. MS m/e 289 (M+H)⁺; ¹H NMR (DMSO-d₆) δ 1.22-1.68 (br m, 2.5H), 1.73-1.89 (br m, 1.5H), 2.48 (s, 3H), 2.86-3.14 (br m, 1.5H), 3.16-3.24 (br m, 0.5H), 3.49-3.71 (br m, 3H), 4.73-4.84 (br m, 0.5H), 4.97-5.03 (br m, 0.5H), 7.76-7.82 (br m, 1H), 7.90 (br d, 1H).

### EXAMPLE 115

### 1-{[2-methyl-6-(trifluoromethyl)-3-pyridinyl]carbonyl}-4-piperidinol

The desired product was prepared by substituting 2-methyl-6-(trifluoromethyl)nicotinic acid for 6-methylnicotinic acid and 4-piperidinol for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 289 (M+H)⁺; ¹H NMR (DMSO-d₆) δ 1.21-1.48 (br m, 2H), 1.66 (br s, 1H), 1.82 (br s, 1H), 2.47 (br s, 3H), 3.02 (br t, 1H), 3.27 (br s, 1H), 3.71-3.79 (m, 1H), 4.04 (br s, 1H), 4.79 (d, 1H), 7.78 (d, 1H), 7.92 (d, 1H).

### EXAMPLE 116

### 1-{[2-methyl-6-(trifluoromethyl)-3-pyridinyl]carbonyl}-3-piperidinecarboxamide

The desired product was prepared by substituting 2-methyl-6-(trifluoromethyl)nicotinic acid for 6-methylnicotinic acid and nipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 316 (M+H)⁺; ¹H NMR (DMSO-d₆) δ 1.26-1.70 (br m, 2H), 1.77-1.99 (br m, 1H), 2.20-2.42 (br m, 1H), 2.48 (br s, 3H), 2.54 (s, 1H), 2.87-3.07 (br m, 1.5H), 3.13-3.28 (br m, 1.5H), 4.20-4.29 (m, 0.5H), 4.45 (br s, 0.5H), 6.80 (br s, 0.5H), 6.89 (br s, 0.5H), 7.24 (br s, 0.5H), 7.41 (br s, 0.5H), 7.78 (t, 1H), 7.91 (d, 1H).

### EXAMPLE 117

### 1-{[2-methyl-6-(trifluoromethyl)-3-pyridinyl]carbonyl}-4-piperidinecarboxamide

The desired product was prepared by substituting 2-methyl-6-(trifluoromethyl)nicotinic acid for 6-methylnicotinic acid and isonipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 316 (M+H)⁺; ¹H NMR (DMSO-d₆) δ 1.33-1.70 (br m, 3H), 1.79-1.90 (br m, 1H), 2.31-2.41 (br m, 1H), 2.48 (br s, 3H), 2.54 (s, 1H), 2.85-2.93 (m, 1H), 3.04 (br t, 1H), 4.48 (br d, 1H), 6.79 (br s, 1H), 7.27 (br s, 1H), 7.79 (d, 1H), 7.92 (br d, 1H).

### EXAMPLE 118

### N,N-diethyl-1-{[2-methyl-6-(trifluoromethyl)-3-pyridinyl]carbonyl}-3-piperidinecarboxamide

The desired product was prepared by substituting 2-methyl-6-(trifluoromethyl)nicotinic acid for 6-methylnicotinic acid and N,N-diethylnipecotamide for 2-methylpyrrolidine in Example 1. After workup the crude compound was purified by HPLC on a C—I 8 column with a solvent system increasing in gradient over 50 minutes from 5% to 100% acetonitrile/water containing 0.01% TFA to provide the desired product as the trifluoroacetate salt. MS m/e 372 (14+H)⁺; ¹H NMR (DMSO-d₆) δ 0.9 (t, 3H), 1.02 (t, 1.5H), 1.16 (t, 1.5H), 1.37-1.70 (br m, 2H), 1.72-1.86 (br m, 2H), 2.46 (br s, 3H), 2.74 (br s, 1H), 2.89-3.12 (br m, 2H), 3.14-3.31 (br s, 5H), 4.35-4.50 (br m, 1H), 7.74-7.84 (m, 1H), 7.89-8.16 (br m, 1H).

### EXAMPLE 119

### 8-{[2-methyl-6-(trifluoromethyl)-3-pyridinyl]carbonyl}-1,4-dioxa-8-azaspiro[4.5]decane

The desired product was prepared by substituting 2-methyl-6-(trifluoromethyl)nicotinic acid for 6-methylnicotinic acid and 1,4-dioxa-8-azaspiro[4.5]decane for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 332 (M+H)⁺; ¹H NMR (DMSO-d₆) δ 1.58 (br d, 2H), 1.72 (br s, 2H), 2.48 (s, 3H), 3.18-3.32 (m, 2H), 3.74 (br d, 2H), 3.86-3.96 (m, 4H), 7.79 (d, 1H), 7.99 (d, 1H).

### EXAMPLE 120

### 4-{[2-methyl-6-(trifluoromethyl)-3-pyridinyl]carbonyl}-1-piperazinecarbaldehyde

The desired product was prepared by substituting 2-methyl-6-(trifluoromethyl)nicotinic acid for 6-methylnicotinic acid and 1-formylpiperidine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 302 (M+H)⁺; ¹H NMR (DMSO-d₆) δ 2.48 (s, 3H), 3.15 (t, 1H), 3.21 (t, 1H), 3.33-3.38 (m, 2H), 3.48-3.54 (m, 2H), 3.65 (br t, 1H), 3.71 (br s, 1H), 7.82 (d, 1H), 7.98 (d, 1H), 8.07 (d, 1H).

### EXAMPLE 121

### 1-acetyl-4-{[2-methyl-6-(trifluoromethyl)-3-pyridinyl]carbonyl}piperazine

The desired product was prepared by substituting 2-methyl-6-(trifluoromethyl)nicotinic acid for 6-methylnicotinic acid and 1-acetylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 316 (M+H)⁺; ¹H NMR (DMSO-d₆) δ 2.01 (d, 3H), 2.48 (s, 3H), 3.14 (t, 1H), 3.20 (t, 1H), 3.37-3.42 (m; 2H), 3.54-3.58 (m, 2H), 3.64 (t, 1H), 3.71 (t, 1H), 7.81 (d, 1H), 7.98 (t, 1H).

### EXAMPLE 122

### 2-(4-{[2-methyl-6-(trifluoromethyl)-3-pyridinyl]carbonyl}-1-piperazinyl)ethanol

The desired product was prepared by substituting 2-methyl-6-(trifluoromethyl)nicotinic acid for 6-methylnicotinic acid and 2-(1-piperazinyl)ethanol for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt MS m/e 318 (M+H)⁺; ¹H NMR (DMSO-d₆) δ 2.52 (br s, 3H), 2.54 (s, 1H), 3.22 (br s, 4H), 3.40-3.54 (br m, 3H), 3.56-3.78 (br m, 4H), 4.58 (br s, 0.5H), 5.37 (br s, 0.5H), 7.48 (d, 1H), 8.02 (d, 1H).

### EXAMPLE 123

### 2-[2-(4-{[2-methyl-6-(trifluoromethyl)-3-pyridinyl]carbonyl}-1-piperazinyl)ethoxy]ethanol

The desired product was prepared by substituting 2-methyl-6-(trifluoromethyl)nicotinic acid for 6-methylnicotinic acid and 2-[2-(1-piperazinyl)ethoxy]ethanol for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 362 (M+H)⁺; ¹H NMR (DMSO-d₆) δ 2.52 (s, 3H), 2.54 (s, 1H), 3.18 (br s, 2H), 3.46-3.51 (m, 3H), 3.52-3.57 (m, 4H), 3.63 (br s, 2H), 3.75 (br s, 3H), 4.58 (br s, 2H), 7.84 (d, 1H), 8.01 (d, 1H).

### EXAMPLE 124

### 1-benzyl-4-{[2-methyl-6-(trifluoromethyl)-3-pyridinyl]carbonyl}piperazine

The desired product was prepared by substituting 2-methyl-6-(trifluoromethyl)nicotinic acid for 6-methylnicotinic acid and 1-benzylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 364 (M+H)⁺; ¹H NMR (DMSO-d₆) δ 2.50-2.58 (m, 3H), 3.20 (br s, 6H), 4.12-4.82 (br m, 4H), 7.47 (br s, 5H), 7.84 (d, 1H), 8.00 (d, 1H).

### EXAMPLE 125

### 1-(4-fluorophenyl)-4-{[2-methyl-6-(trifluoromethyl)-3-pyridinyl]carbonyl}piperazine

The desired product was prepared by substituting 2-methyl-6-(trifluoromethyl)nicotinic acid for 6-methylnicotinic acid and 1-(4-fluorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt MS m/e 368 (M+H)⁺; ¹H NMR (DMSO-d₆) δ 2.51 (s, 3H), 3.02 (br s, 2H), 3.19 (br s, 2H), 3.30 (br s, 2H), 3.82 (br s, 2H), 6.95-7.00 (m, 2H), 7.04-7.10 (m, 2H), 7.81 (d, 1H), 7.99 (d, 1H).

### EXAMPLE 126

### 1-methyl-4-{[2-methyl-6-(trifluoromethyl)-3-pyridinyl]carbonyl}-1,4-diazepane

The desired product was prepared by substituting 2-methyl-6-(trifluoromethyl)nicotinic acid for 6-methylnicotinic acid and 1-methyl-1,4-diazepane for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 302 (M+H)⁺; ¹H NMR (DMSO-d₆) δ 1.94-2.04 (br m, 2H), 2.52 (s, 2H), 2.54 (s, 1H), 2.80 (s, 1H), 2.89 (s, 2H), 3.14-3.65 (br m, 8H), 7.81-7.89 (m, 1H), 8.00-8.08 (m, 1H).

### EXAMPLE 127

### 1-{[4-(trifluoromethyl)-3-pyridinyl]carbonyl}-4-piperidinecarboxamide

The desired product was prepared by substituting (4-trifluoromethyl)nicotinic acid for 6-methylnicotinic acid and isonipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 302 (M+H)⁺; ¹H NMR (DMSO-d₆) δ 1.26-1.77 (m, 3H), 1.83 (d, 1H), 2.34-2.45 (m, 1H), 2.82-3.14 (m, 2H), 3.25-3.41 (br m, 1H), 4.45 (t, 1H), 6.71-6.85 (br m, 1H), 7.20-7.33 (br m, 1H), 7.85 (t, 1H), 8.77 (d, 1H), 8.90 (t, 1H).

### EXAMPLE 128

### 1-methyl-4-{[4-(trifluoromethyl)-3-pyridinyl]carbonyl}piperazine

The desired product was prepared by substituting 4-(trifluoromethyl)nicotinic acid for 6-methylnicotinic acid and 1-methylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 274 (M+H)⁺; ¹H NMR (DMSO-d₆) δ 2.09-2.16 (br m, 1H), 2.19 (s, 3H), 2.24-2.35 (br m, 2H), 2.42-2.48 (br m, 1H), 3.13 (br d, 2H), 3.65 (br d, 2H), 7.85 (d, 1H), 8.77 (s, 1H), 8.91 (d, 1H).

### EXAMPLE 129

### 1-ethyl-4-{[4-(trifluoromethyl)-3-pyridinyl]carbonyl}piperazine

The desired product was prepared by substituting 4-(trifluoromethyl)nicotinic acid for 6-methylnicotinic acid and 1-ethylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 288 (M+H)⁺; ¹H NMR (DMSO-d₆) δ 0.99 (t, 3H), 2.13-2.21 (br m, 1H), 2.29-2.40 (m, 5H), 3.04-3.11 (br m, 1H), 3.14-3.21 (br m, 1H), 3.66 (br d, 2H), 7.86 (d, 1H), 8.77 (s, 1H), 8.91 (d, 1H).

### EXAMPLE 130

### 2-(4-{[4-(trifluoromethyl)-3-pyridinyl]carbonyl}-1-piperazinyl)ethanol

The desired product was prepared by substituting 4-(trifluoromethyl)nicotinic acid for 6-methylnicotinic acid and 2-(1-piperazinyl)ethanol for 2-methylpyrrolidine. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 304 (M+H)⁺; ¹H NMR (DMSO-d₆) δ 2.25 (br t, 1H), 2.37-2.47 (m, 4H), 2.53-2.61 (br m, 1H), 3.03-3.11 (m, 2H), 3.13-3.21 (br m, 1H), 3.49 (q, 2H), 3.55-3.63 (br m, 1H), 3.66-3.73 (br m, 1H), 4.39 (t, 1H), 7.85 (d, 1H), 8.71 (s, 1H), 8.91 (d, 1H).

### EXAMPLE 131

### 1-phenyl-4-{[4-(trifluoromethyl)-3-pyridinyl]carbonyl}piperazine

The desired product was prepared by substituting 4-(trifluoromethyl)nicotinic acid for 6-methylnicotinic acid and 1-phenylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 336 (M+H)⁺; ¹H NMR (DMSO-d₆) δ 3.00 (br s, 1H), 3.06-3.23 (br m, 4H), 3.48-3.61 (br m, 1H), 3.77-3.84 (m, 2H), 6.82 (t, 1H), 6.93-6.98 (m, 2H), 7.20-7.26 (m, 2H), 7.88 (d, 1H), 8.85 (s, 1H), 8.93 (d, 1H).

### EXAMPLE 132

### 1-(4-chlorophenyl)-4-{[4-(trifluoromethyl)-3-pyridinyl]carbonyl}piperazine

The desired product was prepared by substituting 4-(trifluoromethyl)nicotinic acid for 6-methylnicotinic acid and 1-(4-chlorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 370 (M+H)⁺; ¹H NMR (DMSO-d₆) δ 3.11 (br s, 2H), 3.17-3.23 (br m, 2H), 3.40 (br s, 0.5H), 3.49-3.60 (br m, 0.5H), 3.78-3.84 (m, 2H), 4.00 (s, 1H), 7.09-7.14 (m, 1H), 7.20 (s, 1H), 7.24 (dd, 1H), 7.44 (t, 1H), 7.89 (d, 1H), 8.86 (s, 1H), 8.94 (d, 1H).

### EXAMPLE 133

### 1-[3-(trifluoromethyl)phenyl]-4-{[4-(trifluoromethyl)-3-pyridinyl]carbonyl}piperazine

The desired product was prepared by substituting 4-(trifluoromethyl)nicotinic acid for 6-methylnicotinic acid and 1-[3-(trifluoromethyl)phenyl]piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 404 (+H)⁺; ¹H NMR (DMSO-d₆) δ 3.11 (br s, 2H), 3.17-3.23 (br m, 2H), 3.40 (br s, 0.5H), 3.49-3.60 (br m, 0.5H), 3.78-3.84 (m, 2H), 4.00 (s, 1H), 7.09-7.14 (m, 1H), 7.20 (s, 1H), 7.24 (dd, 1H), 7.44 (t, 1H), 7.89 (d, 1H), 8.86 (s, 1H), 8.94 (d, 1H).

### EXAMPLE 134

### 6-methyl-3-[(2-methyl-1-pyrrolidinyl)carbonyl]-2-pyridinol

The desired product was prepared by substituting 2-hydroxy-6-methylnicotinic acid for 6-methylnicotinic acid in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 221 (M+H)⁺; ¹H NMR (DMSO-d₆) δ 0.90 (d, 1H), 1.18 (d, 2H), 1.47-1.58 (m, 1H), 1.65-1.76 (m, 1H), 1.79-2.03 (m, 2H), 2.19 (d, 3H), 3.19-3.27 (m, 0.8H), 3.34-3.48 (m, 1.2H), 3.88-3.96 (m, 0.3H), 4.03-4.11 (m, 0.7H), 6.03 (t, 1H), 7.32-7.38 (m, 1H).

### EXAMPLE 135

### 3-{[4-(2-hydroxyethyl)-1-piperazinyl]carbonyl}-6-methyl-2-pyridinol

The desired product was prepared by substituting 2-hydroxy-6-methylnicotinic acid for 6-methylnicotinic and 2-(1-piperazinyl)ethanol for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 266 (M+H)⁺; ¹H NMR (DMSO-d₆) δ 2.19 (s, 3H), 2.35-2.39 (br m, 2H), 2.55 (br t, 2H), 2.98 (br t, 2H), 3.19 (br t, 2H), 3.47-3.56 (m, 4H), 4.38 (br s, 1H), 6.04 (d, 1H), 7.36 (d, 1H).

### EXAMPLE 136

### 1-[(2-hydroxy-6-methyl-3-pyridinyl)carbonyl]-4-piperidinecarboxamide

The desired product was prepared by substituting 2-hydroxy-6-methylnicotinic acid for 6-methylnicotinic and isonipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 264 (M+H)⁺; ¹H NMR (DMSO-d₆) δ 1.53-1.68 (br m, 3H), 1.74 (d, 1H), 2.19 (s, 3H), 2.70 (t, 1H), 2.87-3.02 (m, 2H), 3.45 (d, 1H), 4.39 (d, 1H), 6.03 (d, 1H), 6.61 (br s, 0.5H), 6.74 (br s, 1H), 7.11 (br s, 0.5H), 7.23 (br s, 1H), 7.34 (d, 1H).

### EXAMPLE 137

### 6-methyl-3-[(4-methyl-1-piperazinyl)carbonyl]-2-pyridinol

The desired product was prepared by substituting 2-hydroxy-6-methylnicotinic acid for 6-methylnicotinic and 1-methylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 236 (M+H)⁺; ¹H NMR (DMSO-d₆) δ 2.19 (s, 3H), 2.20 (s, 3H), 2.31 (br d, 4H), 3.21 (br t, 2H), 3.54 (br t, 2H), 6.04 (dd, 1H), 7.36 (d, 1H).

### EXAMPLE 138

### 6-methyl-3-[(4-phenyl-1-piperazinyl)carbonyl]-2-pyridinol

The desired product was prepared by substituting 2-hydroxy-6-methylnicotinic acid for 6-methylnicotinic and 1-phenylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 298 (+H)⁺; ¹H NMR (DMSO-d₆) δ 2.21 (s, 3H), 3.10-3.20 (m, 4H), 3.37 (br t, 2H), 3.69 (br t, 2H), 6.07 (dd, 1H), 6.80 (t, 1H), 6.94 (d, 2H), 7.19-7.25 (m, 2H), 7.42 (d, 1H).

### EXAMPLE 139

### 3-[(4-benzyl-1-piperazinyl)carbonyl]-6-methyl-2-pyridinol

The desired product was prepared by substituting 2-hydroxy-6-methylnicotinic acid for 6-methylnicotinic and 1-benzylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 312 (M+H)⁺; ¹H NMR (DMSO-d₆) δ 2.18 (s, 3H), 2.31-2.39 (m, 4H), 2.44 (t, 1H), 2.93 (t, 1H), 3.22 (br t, 2H), 3.54 (br t, 2H), 6.03 (d, 1H), 7.27-7.33 (m, 5H), 7.36 (d, 1H).

### EXAMPLE 140

### 3-{[4-(4-chlorophenyl)-1-piperazinyl]carbonyl}-6-methyl-2-pyridinol

The desired product was prepared by substituting 2-hydroxy-6-methylnicotinic acid for 6-methylnicotinic and 1-(4-chlorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 332 (M+H)⁺; ¹H NMR (DMSO-d₆) δ 2.21 (s, 3H), 3.10-3.20 (m, 4H), 3.35 (br t, 2H), 3.68 (br t, 2H), 6.07 (d, 1H), 6.95 (d, 2H), 7.24 (d, 2H), 7.43 (d, 1H).

### EXAMPLE 141

### 5-chloro-3-[(3-methyl-1-piperidinyl)carbonyl]-2-pyridinol

The desired product was prepared by substituting 2-hydroxy-5-chloronicotinic acid for 6-methylnicotinic and 3-methylpiperidine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 255 (M+H)⁺; ¹H NMR (DMSO-d₆) δ 0.76 (d, 1.3H), 0.90 (d, 1.7H), 1.06-1.18 (br m, 1H), 1.32-1.78 (br m, 5H), 2.40-2.46 (m, 0.5H), 2.59-2.72 (m, 1H), 2.89-2.98 (m, 0.5H), 4.18 (d, 0.5H), 4.27 (d, 0.5H), 7.50 (s, 1H), 7.70 (br s, 1H).

### EXAMPLE 142

### (3R)-1-{[5-(2,5-dimethylphenyl)-3-pyridinyl]carbonyl}-N,N-dimethyl-3-pyrrolidinamine

### EXAMPLE 142A

### (3R)-1-[(5-bromo-3-pyridinyl)carbonyl]-N,N-dimethyl-3-pyrrolidinamine

The desired product was prepared by substituting (3R)-N,N-dimethyl-3-pyrrolidinamine for 2-methylpyrrolidine in Example 30.

### EXAMPLE 142B

### (3R)-1-{[5-(2,5-dimethylphenyl)-3-pyridinyl]carbonyl}-N,N-dimethyl-3-pyrrolidinamine

The desired product was prepared by substituting Example 142A for Example 30 in Example 59. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 324 (M+H)⁺; ¹H NMR (DMSO-d₆) δ 2.05-2.18 (m, 1H), 2.17-2.41 (m, 7H), 2.71-2.95 (m, 6H), 3.52-3.80 (m, 3H), 3.85-4.01 (m, 2H), 7.10 (s, 1H), 7.17 (d, 1H), 7.24 (d, 1H), 7.92 (t, 1H), 8.66 (br s, 1H), 8.72 (d, 1H).

### EXAMPLE 143

### (3S)-1-{[5-(2,5-dimethylphenyl)-3-pyridinyl]carbonyl}-N,N-dimethyl-3-pyrrolidinamine

### EXAMPLE 143A

### (3S)-1-[(5-bromo-3-pyridinyl)carbonyl]-N,N-dimethyl-3-pyrrolidinamine

The desired product was prepared by substituting (3S)-N,N-dimethyl-3-pyrrolidinamine for 2-methylpyrrolidine in Example 30.

### EXAMPLE 143B

### (3S)-1-{[5-(2,5-dimethylphenyl)-3-pyridinyl]carbonyl}-N,N-dimethyl-3-pyrrolidinamine

The desired product was prepared by substituting Example 143A for Example 30 in Example 143B. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 324 (M+H)⁺; ¹H NMR (DMSO-d₆) δ 2.06-2.18 (m, 1H), 2.16-2.39 (m, 7H), 2.73-2.96 (m, 6H), 3.51-3.80 (m, 3H), 3.84-4.00 (m, 2H), 7.11 (s, 1H), 7.17 (d, 1H), 7.24 (d, 1H), 7.92 (t, 1H), 8.64 (br s, 1H), 8.72 (d, 1H).

### EXAMPLE 144

### (2R)-1-[(6-methyl-3-pyridinyl)carbonyl]-2-piperidinecarboxamide

The desired product was prepared by substituting (2R)-2-piperidinecarboxamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 248 (M+H)⁺; ¹H NMR (DMSO-d₆) δ 1.24-1.77 (m, 5H), 2.02-2.33 (m, 1H), 2.60 (s, 3H), 2.77-3.09 (br m, 0.5H), 3.17-3.50 (m, 1H), 4.11 (br s, 0.25H), 4.42 (br s, 0.25H), 5.06 (br s, 1H), 7.26 (br s, 1H), 7.46 (s, 1H), 7.60 (d, 1H), 8.00 (brd, 1H), 8.63 (d, 1H).

### EXAMPLE 145

### (2S)-1-[(6-methyl-3-pyridinyl)carbonyl]-2-piperidinecarboxamide

The desired product was prepared by substituting (2S)-2-piperidinecarboxamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 248 (M+H)⁺; ¹H NMR (DMSO-d₆) δ 1.21-1.76 (m, 5H), 2.00-2.30 (m, 1H), 2.60 (s, 3H), 2.76-3.10 (br m, 0.5H), 3.16-3.50 (m, 1H), 4.13 (br s, 0.25H), 4.40 (br s, 0.25H), 5.05 (br s, 1H), 7.26 (br s, 1H), 7.46 (s, 1H), 7.60 (d, 1H), 7.98(br d, 1H), 8.64 (br d, 1H).

### EXAMPLE 146

### (3R)-N-(3-furylmethyl)-1-[(6-methyl-3-pyridinyl)carbonyl]-3-pyrrolidinamine

The desired product was prepared by substituting tert-butyl (3R)-3-pyrrolidinylcarbamate for 2-methylpyrrolidine in Example 1. After workup (tert-butyl (3R)-1-[(6-methyl-3-pyridinyl)carbonyl]-3-pyrrolidinylcarbamate was obtained. This was treated with a (1:1) mixture of triflouroacetic acid/dichloromethane at room temperature with stirring for 1 hour and concentrated in vacuo. The residue was dissolved in a mixture of dichloromethane/acetic acid (10:1), treated with 3-furaldehyde (3 equivalents) in the presence of 4 Å molecular sieves and shaken for 2 hours. Polystyrylmethyltrimethylammonium cyanoborohydride resin (4 equivalents) was added and the mixture was shaken for 16 hours. The reaction mixture was filtered and the filtrate was concentrated in vacuo. The crude product was purified by HPLC using a C-18 column and a solvent system varying over 50 minutes in a gradient from 5% to 100% acetonitrile/water containing 0.01% TFA then lyophilized to give the desired product as trifluoroacetic acid salt. This was dissolved in (1:4) methanol/dichloromethane and shaken with MP carbonate resin (3 eqivalents) for 3 hours, dissolved in dioxane, and treated dropwise an excess of 2.0 M HCl in diethyl ether. The precipitate was isolated by filtration to provide the desired product as the hydrochloride salt. MS m/e 248 (M+H)⁺; ¹H NMR (DMSO-d₆) δ 2.26 (br s, 2H), 2.64 (s, 3H), 3.45-3.61 (m, 1H), 3.66-3.86 (m, 5H), 3.97-4.16 (m, 2H), 6.77 (d, 1H), 7.60-7.91 (m, 3H), 8.20 (dd, 1H), 8.81 (d, 1H).

### EXAMPLE 147

### (3R)-N,N-dimethyl-1-{[2-methyl-6-(trifluoromethyl)-3-pyridinyl]carbonyl}-3-pyrrolidinamine

The desired product was prepared by substituting 2-methyl-6-(trifluoromethyl)nicotinic acid for 6-methylnicotinic acid and (3R)-N,N-dimethyl-3-pyrrolidinamine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 302 (M+H)⁺; ¹H NMR (DMSO-d₆) δ 2.17-2.33 (m, 2H), 2.52 (d, 2H), 2.65 (d, 1H), 2.70-2.85 (m, 6H), 3.18-3.30 (m, 1H), 3.31-3.43 (m, 1H), 3.45-3.66 (m, 1H), 3.74-4.03 (m, 2H), 7.81 (d, 0.4H), 7.84 (d, 0.6H), 8.01 (d, 0.4H), 8.07 (d, 0.6H).

### EXAMPLE 148

### (3R)-1-[(2-chloro-6-methyl-3-pyridinyl)carbonyl]-N,N-dimethyl-3-pyrrolidinamine

The desired product was prepared by substituting 2-chloro-6-methylnicotinic acid for 6-methylnicotinic acid and (3R)-N,N-dimethyl-3-pyrrolidinamine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 267.9 (M+H)⁺; ¹H NMR (DMSO-d₆) δ 2.14-2.43 (m, 2H), 2.50 (s, 3H), 2.66 (d, 1H), 2.69-2.86 (m, 5H), 3.18-3.56 (m, 2H), 3.57-4.01 (m, 3H), 7.39 (dd, 1H), 7.83 (dd, 1H).

### EXAMPLE 149

### (3R)-N,N-dimethyl-1-{[6-(1H-pyrazol-1-yl)-3-pyridinyl]carbonyl}-3-pyrrolidinamine

The desired product was prepared by substituting 6-pyrazolylnicotinic acid for 6-methylnicotinic and (3R)-N,N-dimethyl-3-pyrrolidinamine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 286 (M+H)⁺; ¹H NMR (DMSO-d₆) δ 2.13-2.44 (m, 2H), 2.64-2.89 (br m, 6H), 3.46-4.01 (m, 5H), 6.63 (q, 1H), 7.89 (d, 1H), 7.99 (d, 1H), 8.18 (brd, 1H), 8.66 (d, 2H).

### EXAMPLE 150

### (3R)-N,N-dimethyl-1-{[6-(trifluoromethyl)-3-pyridinyl]carbonyl}-3-pyrrolidinamine

The desired product was prepared by substituting 6-(trifluoromethyl)nicotinic acid for 6-methylnicotinic and (3R)-N,N-dimethyl-3-pyrrolidinamine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 288 (M+H)⁺; ¹H NMR (DMSO-d₆) δ 2.15-2.43 (m, 2H), 2.65-2.90 (br m, 6H), 3.48-4.01 (m, 5H), 8.02 (dd, 1H), 8.21-8.31 (m, 1H), 8.92 (dd, 1H).

### EXAMPLE 151

### (3R)-N,N-dimethyl-1-(3-pyridinylcarbonyl)-3-pyrrolidinamine

The desired product was prepared by substituting nicotinic acid for 6-methylnicotinic and (3R)-N,N-dimethyl-3-pyrrolidinamine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 220 (M+H)⁺; ¹H NMR (DMSO-d₆) δ 2.20-2.43 (m, 2H), 2.65-2.86 (m, 6H), 3.47-3.60 (m, 1H), 3.62-3.99 (m, 4H), 7.83-7.84 (m, 1H), 8.42 (t, 1H), 8.88 (t, 1H), 8.98 (d, 1H).

### EXAMPLE 152

### 1-[(6-methyl-3-pyridinyl)carbonyl]-3-pyrrolidinecarboxamide

The desired product was prepared by substituting 3-pyrrolidinecarboxamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 234 (M+H)⁺; ¹H NMR (DMSO-d₆) δ 1.89-2.22 (m, 2H), 2.71 (s, 3H), 2.88-3.08 (m, 1H), 3.42-3.76 (m, 4H), 6.99 (br d, 1H), 7.52 (br d, 1H), 7.82 (dd, 1H), 8.37-8.44 (m, 1H), 8.87 (dd, 1H).

### EXAMPLE 153

### 2-methyl-6-[(2-methyl-1-pyrrolidinyl)carbonyl]pyridine

The desired product was prepared by substituting 6-methylpicolinic acid for 6-methylnicotinic in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 205 (M+H)⁺; ¹H NMR (DMSO-d₆) δ 0.97 (d, 1.2H), 1.36 (d, 1.8H), 1.58-1.70 (m, 1H), 1.74-1.85 (m, 1H), 1.90-2.03 (m, 1H), 2.03-2.15 (m, 1H), 2.66 (s, 3H), 3.54-3.64 (m, 0.6H), 3.68-3.84 (m, 1.4H), 4.33-4.42 (m, 0.6H), 4.61-4.69 (m, 0.4H), 7.16 (t, 1H), 7.52 (t, 1H), 7.61-7.68 (m, 1H).

### EXAMPLE 154

### 3-[(4-ethyl-1-piperazinyl) carbonyl]-6-methyl-2-pyridinol

The desired product was prepared by substituting 2-hydroxy-6-methylnicotinic acid for 6-methylnicotinic and 1-ethylpiperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 250 (M+H)⁺; ¹H NMR (DMSO-d₆) δ 1.00 (t, 3H), 2.19 (s, 3H), 2.29-2.41 (m, 6H), 3.21 (br t, 2H), 3.54 (br t, 2H), 6.04 (d, 1H), 7.36 (d, 1H).

### EXAMPLE 155

### 1-[(5-methyl-3-pyridinyl)carbonyl]-3-piperidinecarboxamide

A stirred solution of 5-methylnicotinic acid (8 mmol) in (9:1) acetonitrile/methylenechloride (20 mL) under nitrogen was treated with N-hydroxysuccinimide (9.5 mmol). The mixture was stirred at room temperature until all solids dissolved. The solution was treated with 1-(3-dimethylaminopropyl)-3-ethylcarbodiimide hydrochloride (EDC) (8.8 mmol), stirred at room temperature overnight, and concentrated in vacuo. The residue was crystallized from ethyl acetate/hexanes to provide the N-hydroxysuccinimide ester.

A solution of the N-hydroxysuccinimide ester (0.884 mmol) and nipecotamide (0.884 mmol) in dichloromethane (9 mL) was heated to reflux for 4 hours and stirred at room temperature overnight. The reaction mixture was twice shaken with MP-carbonate resin (1 g) for one hour and filtered. The filtrate was concentrated in vacuo and the residue was crystallized from ethyl acetate/hexanes to provide the desired product. MS m/e 248.1 (M+H)⁺; ¹H NMR (DMSO-d₆) δ 1.30-1.52 (br m, 1H), 1.52-1.82 (br m, 1H), 1.82-2.00 (br m, 1H), 2.2-2.35 (br m, 1H), 2.32 (s, 3H), 2.75-2.90 (br m, 1H), 2.90-3.28 (m, 1H), 3.40-3.56 (br m, 1H), 4.20-4.35 (brd, 0.5H), 4.35-4.53 (br d, 0.5H), 6.80-6.95 (br m, 1H), 7.23-7.46 (br d, 1H), 7.62 (br s, 1H), 8.38 (br d, 1H), 8.50 (br d, 1H).

### EXAMPLE 156

### (3R)-N,N-dimethyl-1-[(2-phenoxy-3-pyridinyl)carbonyl]-3-pyrrolidinamine

The desired product was prepared by substituting procedure 2-phenoxynicotinic acid for 5-methylnicotinic acid and (3R)-N,N-dimethyl-3-pyrrolidinamine for nipecotamide in Example 155. The free base was dissolved in diethyl ether and adjusted to pH 1 with 1 M HCl in diethyl ether. The precipitate was filtered and dried to provide the desired product as the hydrochloride salt. MS m/e 312 (M+H)⁺; ¹H NMR (DMSO-d₆) δ 2.15-2.44 (br m, 1H), 2.66-2.83 (br m, 6H), 3.40-3.62 (br m, 1H), 3.65-4.05 (br m, 5H), 7.11-7.28 (m, 4H), 7.35-7.46 (m, 2H), 7.85-7.95 (m, 1H), 8.16-8.22 (m, 1H), 11.08-11.27 (br m, 1H).

### EXAMPLE 157

### 1-[(6-methyl-3-pyridinyl)carbonyl]-3-pyrrolidinecarboxylic acid

A solution of 6-methylnicotinic acid N-hydroxysuccinimide ester (1 mmol, prepared according to the procedure described in Example 155), 3-pyrrolidinecarboxylic acid (1.19 mmol), and triethylamine (3 mmol) in dichloromethane (8 mL) was stirred at room temperature overnight. The reaction mixture was concentrated in vacuo and purified by HPLC using a C-18 column and a solvent system varying in a gradient of 10% to 90% acetonitrile/water containing 0.1% TFA and lyophilized to provide the desired compound as the TFA salt. MS m/e 235 (14+H)⁺; ¹H NMR (DMSO-d₆) δ 1.97-2.22 (m, 2H), 2.56 (s, 3H), 3.03-3.17 (m, 1H), 3.43-3.77 (m, 4H), 7.48 (dd, 1H), 7.97-8.05 (m, 1H), 8.66-8.70 (m, 1H).

### EXAMPLE 158

### methyl 1-[(6-methyl-3-pyridinyl)carbonyl]-3-pyrrolidinecarboxylate

A solution of 6-methylnicotinc acid N-hydroxysuccinimide ester (1 mmol), 3-pyrrolidinecarboxylic acid (1.19 mmol), and triethylamine (3 mmol) in dichloromethane (8 mL) was stirred at room temperature overnight. The reaction mixture was concentrated in vacuo, purified by HPLC on a C-18 column using a solvent system varying in a gradient of 10% to 90% acetonitrile/water containing 0.1% TFA, and lyophilized to provide 6-methylnicotinyl-(3-pyrrolidinecarboxylic acid)amide. The acid was dissolved in methanol, treated with several drops of concentrated HCl, heated to reflux for 2 hours, cooled to room temperature, concentrated in vacuo, dissolved in dichloromethane, washed with sodium bicarbonate, water, and brine, dried (Na₂SO₄), filtered, and concentrated in vacuo. The concentrate was recrystallized from hot ethyl acetate to provide the desired product. MS m/e 248.9 (M+H)⁺; ¹H NMR (DMSO-d₆) δ 1.94-2.28 (m, 2H), 2.69 (s, 3H), 3.00-3.28 (m, 1H), 3.44-3.91 (m, 7H), 7.77 (dd, 1H), 8.32-8.39 (m, 1H), 8.84 (dd, 1H).

### EXAMPLE 159

### ethyl 1-[(6-methyl-3-pyridinyl)carbonyl]-3-piperidinecarboxylate

The desired product was prepared by substituting ethyl nipecotate for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 277 (M+H)⁺; ¹H NMR (DMSO-d₆) δ 1.04-1.31 (m, 3H), 1.41-1.82 (m, 3H), 1.90-2.07 (m, 1H), 2.56-2.76 (m, 4H), 3.00-3.65 (br m, 3H), 3.81-4.59 (br m, 3H), 7.47 (d, 1H), 8.20 (s, 1H), 8.74 (s, 1H).

### EXAMPLE 160

### 1-isonicotinoyl-4-piperidinecarboxamide

The desired product was prepared by substituting isonicotinic acid for 6-methylnicotinic and isonipecotamide for 2-methylpyrrolidine in Example 1 After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 234 (M+H)⁺; ¹H NMR (DMSO-d₆) δ 1.29-1.53 (m, 1H), 1.53-1.82 (m, 2H), 1.84-2.01 (m, 1H), 2.25-2.41 (m, 1H), 2.82-3.09 (m, 1.5H), 3.17 (t, 0.5H), 3.37 (t, 1H), 4.20 (d, 0.5H), 4.43 (d, 0.5H), 6.86 (d, 1H), 7.33 (d, 1H), 7.58 (dd, 2H), 8.77 (d, 2H).

### EXAMPLE 161

### 1-isonicotinoyl-3-piperidinecarboxamide

The desired product was prepared by substituting isonicotinic acid for 6-methylnicotinic and nipecotamide for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 234 (M+H)⁺; ¹H NMR (DMSO-d₆) δ 1.42-1.60 (m, 2H), 1.66 (d, 1H), 1.83 (d, 1H), 2.33-2.44 (m, 1H), 2.87 (t, 1H), 3.06 (t, 1H), 3.43 (d, 1H), 4.41 (d, 1H), 6.80 (s, 1H), 7.27 (s, 1H), 7.57 (dd, 2H), 8.76 (dd, 2H).

### EXAMPLE 162

### 4-[(2-methyl-1-pyrrolidinyl)carbonyl]pyridine

The desired product was prepared by substituting isonicotinic acid for 6-methylnicotinic in Example 1. After workup the crude compound was purified by HPLC on a C-18 column with a solvent system increasing in gradient over 50 minutes from 5% to 100% acetonitrile/water containing 0.01% TFA to provide the desired product as the trifluoroacetate salt. MS m/e 191 (M+H)⁺; ¹H NMR (DMSO-d₆) δ 0.85 (d, 0.8H), 1.26 (d, 2.2H), 1.52-1.62 (m, 1H), 1.68-1.79 (m, 1H), 1.82-1.95 (m, 1H), 2.01-2.13 (m, 1H), 3.20-3.29 (m, 0.7H), 3.37-3.45 (m, 0.7H), 3.48-3.60 (m, 0.6H), 3.84-3.92 (m, 0.25H), 4.11-4.21 (m, 0.75H), 7.65 (dd, 2H), 8.77 (dd, 2H).

### EXAMPLE 163

### (3R)-1-isonicotinoyl-N,N-dimethyl-3-pyrrolidinamine

The desired product was prepared by substituting isonicotinic acid for 6-methylnicotinic acid and (3R)-3-(dimethylamino)pyrrolidine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. This was dissolved in dichloromethane and shaken with basic resin MP carbonate for four hours. The resin was removed by filtration and the filtrate was concentrated in vacuo. The free base was dissolved in diethyl ether and treated dropwise with 1.0 M HCl in diethyl ether. The precipitate was isolated by filtration to provide the desired product as the dihydrochloride salt. MS m/e 220 (M+H)⁺; ¹H NMR (DMSO-d₆) δ 2.06-2.20 (m, 1H), 2.24-2.40 (m, 1H), 2.69-2.87 (m, 6H), 3.43-3.62 (m, 2H), 3.64-3.98 (m, 3H), 7.49 (dd, 2H), 8.67-8.73 (dd, 2H).

### EXAMPLE 164

### 1-(4-fluorophenyl)-4-isonicotinoylpiperazine

The desired product was prepared by substituting isonicotinic acid for 6-methylnicotinic acid and (4-fluorophenyl)piperazine for 2-methylpyrrolidine in Example 1. After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. This was dissolved in dichloromethane and shaken with basic resin MP carbonate for four hours. The resin was removed by filtration and the filtrate was concentrated in vacuo. The free base product was dissolved in diethyl ether and treated dropwise with 1.0 M HCl in diethyl ether. The precipitate was isolated by filtration to provide the desired product as the dihydrochloride salt. MS m/e 285.9 (+H)⁺; ¹H NMR (DMSO-d₆) δ 3.07 (br t, 2H), 3.19 (br t, 2H), 3.40 (br t, 2H), 3.78 (br t, 2H), 6.00-7.02 (m, 2H), 7.04-7.11 (m, 2H), 7.61 (dd, 2H), 8.78 (dd, 2H).

### EXAMPLE 165

### 2-methyl-5-[(2-methyl-1-pyrrolidinyl)carbonyl]pyrazine

The desired product was prepared by substituting 5-methyl-2-pyrazinecarboxylic acid for 6-methylnicotinic acid in Example 1. After workup the crude compound was purified by HPLC on a C-18 column with a solvent system increasing in gradient over 50 minutes from 5% to 100% acetonitrile/water containing 0.01% TFA to provide the desired product as the bis(trifluoroacetate) salt. This was dissolved in dichloromethane and shaken with basic resin MP carbonate for four hours. The resin was removed by filtration and the filtrate was concentrated in vacuo. The free base product was dissolved in diethyl ether and treated dropwise with 1.0 M HCk in diethyl ether. The precipitate was isolated by filtration to provide the desired product as the dihydrochloride salt MS m/e 206 (M+H)⁺; ¹H NMR (DMSO-d₆) δ 1.10 (br s, 1H), 1.36 (br d, 2H), 1.61-1.82 (m, 1H), 1.83-2.18 (m, 3H), 2.92 (s, 3H), 3.66-3.81 (br m, 1.4H), 3.91 (br s, 0.6H), 4.42 (br d, 0.7H), 4.78 (br s, 0.3H), 8.82 (s, 1H), 9.05 (s, 1H).

### EXAMPLE 166

### 5-[(2-methyl-1-pyrrolidinyl)carbonyl]pyrimidine

The desired product can be prepared by substituting 5-pyrimidinecarboxylic acid for 6-methylnicotinic acid in Example 1. After workup the crude compound is purified by HPLC on a C-18 column with a solvent system increasing in gradient over 50 minutes from 5% to 100% acetonitrile/water containing 0.01% TFA to provide the product as the trifluoroacetate salt. This dissolved in dichloromethane and shaken with basic resin MP carbonate for four hours. The resin is removed by filtration and the filtrate is concentrated in vacuo. The free base is dissolved in diethyl ether and treated dropwise with 1.0 M HCl in diethyl ether. The precipitate isolated by filtration to provide the desired product as the hydrochloride salt.

### EXAMPLE 167

### 4-methyl-5-[(2-methyl-1-pyrrolidinyl)carbonyl]-2-phenylpyrimidine

The desired product can be prepared by substituting 4-methyl-2-phenyl-5-pyrimidinecarboxylic acid for 6-methylnicotinic acid in Example 1. After workup the crude compound is purified by HPLC on a C-18 column with a solvent system increasing in gradient over 50 minutes from 5% to 100% acetonitrile/water containing 0.01% TFA to provide the desired product as the trifluoroacetate salt. This dissolved in dichloromethane and shaken with basic resin MP carbonate for four hours. The resin is removed by filtration and the filtrate is concentrated in vacuo. The free base is dissolved in diethyl ether and treated dropwise with 1.0 M HCl in diethyl ether. The precipitate isolated by filtration to provide the desired product as the hydrochloride salt.

### EXAMPLE 168

### 2-methyl-5-[(2-methyl-1-pyrrolidinyl)carbonyl]4-phenylpyrimidine

The desired product can be prepared by substituting 2-methyl-4-phenyl-5-pyrimidinecarboxylic acid for 6-methylnicotinic acid in Example 1. After workup the crude compound is purified by HPLC on a C-18 column with a solvent system increasing in gradient over 50 minutes from 5% to 100% acetonitrile/water containing 0.01% TFA to provide the desired product as the trifluoroacetate salt. This dissolved in dichloromethane and shaken with basic resin MP carbonate for four hours. The resin is removed by filtration and the filtrate is concentrated in vacuo. The free base is dissolved in diethyl ether and and treated dropwise with 1.0 M HCl in diethyl ether. The precipitate isolated by filtration to provide the desired product as the hydrochloride salt.

### EXAMPLE 169

### (3S)-1-[(5-methyl-3-pyridinyl)carbonyl]-3-piperidinecarboxamide

The desired product can be prepared by substituting 5-methylnicotinic acid for 6-methylnicotinic acid in Example 57 After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 170

### (3R)-1-[(5-methyl-3-pyridinyl)carbonyl]-3-piperidinecarboxamide

The desired product can be prepared by substituting 5-methylnicotinic acid for 6-methylnicotinic acid in Example 56. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 171

### (3R)-N,N-dimethyl-1-[(5-methyl-3-pyridinyl)carbonyl]-3-pyrrolidinamine

The desired product can be prepared by substituting 5-methylnicotinic acid for 6-methylnicotinic acid in Example 51. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 172

### (3S)-N,N-dimethyl-1-[(5-methyl-3-pyridinyl)carbonyl]-3-pyrrolidinamine

The desired product can be prepared by substituting 5-methylnicotinic acid for 6-methylnicotinic acid in Example 50. After workup the crude compound is purified by HPLC on a C-18 column and a solvent system increasing in gradient over 50 minutes from 5% to 100% acetonitrile/water containing 0.01% TFA. The desired product is obtained as the trifluoroacetate salt.

### EXAMPLE 173

### 1-(4-fluorophenyl)-4-[(5-methyl-3-pyridinyl)carbonyl]piperazine

The desired product can be prepared by substituting 5-methylnicotinic acid for 6-methylnicotinic acid in Example 25. After workup the crude compound is purified by HPLC on a C-18 column with a solvent system increasing in gradient over 50 minutes from 5% to 100% acetonitrile/water containing 0.01% TFA to prepare the desired product as the trifluoroacetate salt.

### EXAMPLE 174

### (2S)-1-[(5-methyl-3-pyridinyl)carbonyl]-2-piperidinecarboxamide

The desired product can be prepared by substituting 5-methylnicotinic acid for 6-methylnicotinic acid in Example 144. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 175

### (2R)-1-[(5-methyl-3-pyridinyl)carbonyl]-2-piperidinecarboxamide

The desired product can be prepared by substituting 5-methylnicotinic acid for 6-methylnicotinic acid in Example 145. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 176

### (3S)-1-[(5-methyl-2-pyrazinyl)carbonyl]-3-piperidinecarboxamide

The desired product can be prepared by substituting (3S)-3-piperazinecarboxamide for 3-methylpyrrolidine in Example 165. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 177

### (3S)-1-(5-pyrimidinylcarbonyl)-3-piperidinecarboxamide

The desired product can be prepared by substituting (3S)-3-piperazinecarboxamide for 3-methylpyrrolidine in Example 166. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 178

### (3R)-N,N-dimethyl-1-[(5-methyl-2-pyrazinyl)carbonyl]-3-pyrrolidinamine

The desired product can be prepared by substituting (3R)-3-dimethylaminopyrrolidine for 3-methylpyrrolidine in Example 165. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 179

### (3R)-N,N-dimethyl-1-(5-pyrimidinylcarbonyl)-3-pyrrolidinamine

The desired product can be prepared by substituting (3R)-3-dimethylaminopyrrolidine for 3-methylpyrrolidine in Example 166. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 180

### 2-methyl-5-[(4-(4-fluorophenyl)piperazinyl)]carbonyl]pyrazine

The desired product can be prepared by substituting 5-methyl-2-pyrazinecarboxylic acid for 2-methylnicotinic acid in Example 25. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 181

### 5-[(4-(4-fluorophenyl)piperazinyl)]carbonyl]pyrimidine

The desired product can be prepared by substituting 5-pirimidinecarboxylic acid for 2-methylnicotinic acid in Example 25. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 182

### (2S)-2-methyl-5-[(2-piperidinecarboxamide)carbonyl]pyrazine

The desired product can be prepared by substituting (2S) 2-piperazinecarboxamide for 3-methylpyrrolidine in Example 165. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

### EXAMPLE 183

### (2S) 5-[(2-piperidinecarboxamide)carbonyl]pyrimidine

The desired product can be prepared by substituting (2S) 2-piperazinecarboxamide for 3-methylpyrrolidine in Example 166. After workup the crude compound is purified by HPLC on a C-18 column with a solvent system increasing in gradient over 50 minutes from 5% to 100% acetonitrile/water containing 0.01% TFA. The desired product is obtained as the trifluoroacetate salt.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of formula (I) 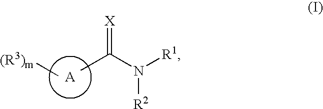
or a therapeutically acceptable salt thereof, wherein
A is an aromatic six-membered ring containing one nitrogen atom wherein the remaining atoms are carbon;
R¹ and R², together with the nitrogen atom to which they are attached, form a five membered-ring wherein the ring can be optionally substituted with one, two, or three substituents independently selected from the group consisting of alkoxyalkyl, alkoxycarbonyl, alkyl, alkylcarbonyl, amino, aminocarbonyl, aryl, arylalkoxycarbonyl, arylalkyl, carboxy, formyl, haloalkyl, heterocycle, (heterocycle)alkyl, hydroxy, hydroxyalkoxyalkyl, hydroxyalkyl, and spiroheterocycle;
R³ at each occurance is independently selected from the group consisting of alkenyl, alkoxy, alkoxyalkyl, alkoxycarbonyl, alkyl, alkylcarbonyl, alkylsulfanyl, amino, aminocarbonyl, aryl, arylalkyl, aryloxy, cyano, cyanoalkyl, cycloalkyl, (cycloalkyl)alkyl, halo, haloalkyl, heterocycle, hydroxy, hydroxyalkyl, and nitro;
X is selected from the group consisting of O, S, and CH₂; and
m is 0-4. (0, 1, 2)

2. The compound of claim 1 wherein X is O and A is a six-membered aromatic ring containing one nitrogen atom wherein the remaining atoms are carbon.

3. (canceled)

4. (canceled)

5. (canceled)

6. (canceled)

7. (canceled)

8. (canceled)

9. (canceled)

10. The compound of claim 2 wherein R¹ and R², together with the nitrogen atom to which they are attached, form a pyrrolidinyl ring.

11. The compound of claim 10 wherein the pyrrolidinyl ring is unsubstituted or substituted with one substituent selected from the group consisting of alkoxyalkyl, alkoxycarbonyl, aminocarbonyl, arylalkoxycarbonyl, carboxy, heterocycle, (heterocycle)alkyl, and hydroxyalkyl.

12. The compound of claim 10 wherein the pyrrolidinyl ring is substituted with one substituent selected from the group consisting of amino, aryl, and arylalkyl.

13. The compound of claim 10 wherein the pyrrolidinyl ring is substituted with one or two alkyl groups.

14. The compound of claim 13 wherein m is 0 or 2.

15. The compound of claim 13 wherein m is 1.

16. The compound of claim 15 wherein R³ is selected from the group consisting of alkyl, halo, and hydroxy.

17. The compound of claim 15 wherein R³ is aryl.

18. The compound of claim 15 wherein R³ is selected from the group consisting of cyanoalkyl, cycloalkyl, (cycloalkyl)alkyl, and heterocycle.

19. The compound of claim 15 wherein R³ is amino.

20. A pharmaceutical composition comprising a compound of claim 1 or a therapeutically acceptable salt thereof, in combination with a therapeutically acceptable carrier.

21. A method for inhibiting angiogenesis in a patient in recognized need of such treatment comprising administering to the patient a therapeutically acceptable amount of a compound of claim 1, or a therapeutically acceptable salt thereof.

22. A method for treating cancer in a patient in recognized need of such treatment comprising administering to the patient a therapeutically acceptable amount of a compound of claim 1, or a therapeutically acceptable salt thereof.
